SEQUENCE LISTING (112 sequences)

SEQ ID NO: 1 - protein, 16 aa, Arabidopsis thaliana
TFDESKETINKEIEEK

SEQ ID NO: 2 - protein, 9 aa, Arabidopsis thaliana
FQAAVDILR

SEQ ID NO: 3 - protein, 16 aa, Arabidopsis thaliana
IKHDIDTETQDIPDAR

SEQ ID NO: 4 - protein, 12 aa, Arabidopsis thaliana
ITLDPEDPAAVK

SEQ ID NO: 5 - protein, 6 aa, Arabidopsis thaliana
VFFDIK

SEQ ID NO: 6 - protein, 18 aa, Arabidopsis thaliana
AQLDELKSDAVEAMESQK

SEQ ID NO: 7 - protein, 15 aa, Arabidopsis thaliana
SDKKGMDLLVAEFEK

SEQ ID NO: 8 - protein, 17 aa, Arabidopsis thaliana
KEDLPKYEENLELSMAK

SEQ ID NO: 9 - protein, 7 aa, Arabidopsis thaliana
YLEELVK

SEQ ID NO: 10 - protein, 10 aa, Arabidopsis thaliana
VSVFLPEEVK

SEQ ID NO: 11 - protein, 13 aa, Arabidopsis thaliana
VVETYEATSAEVK

SEQ ID NO: 12 - protein, 14 aa, Arabidopsis thaliana
EIPVEEVKAEEPAK

SEQ ID NO: 13 - protein, 14 aa, Arabidopsis thaliana
AIAFDEIDKAPEEK

SEQ ID NO: 14 - protein, 10 aa, Arabidopsis thaliana
FPGDDIPIIR

SEQ ID NO: 15 - protein, 11 aa, Arabidopsis thaliana
VGEEVEILGLR

SEQ ID NO: 16 - protein, 16 aa, Arabidopsis thaliana
GSALSALQGTNDEIGR

SEQ ID NO: 17 - protein, 14 aa, Arabidopsis thaliana
LMDAVDEYIPDPVR

SEQ ID NO: 18 - protein, 9 aa, Arabidopsis thaliana
TLVFQFSVK

SEQ ID NO: 19 - protein, 14 aa, Arabidopsis thaliana
FYAISAEFPEFSNK

SEQ ID NO: 20 - protein, 16 aa, Arabidopsis thaliana
FYAISAEFPEFSNKDK

SEQ ID NO: 21 - protein, 14 aa, Arabidopsis thaliana
AVVTVPAYFNDAQR

SEQ ID NO: 22 - protein, 13 aa, Arabidopsis thaliana
EVDEVLLVGGMTR

SEQ ID NO: 23 - protein, 20 aa, Arabidopsis thaliana
GVNPDEAVAMGAAIQGGILR

SEQ ID NO: 24 - protein, 9 aa, Arabidopsis thaliana
LVPYQIVNK

SEQ ID NO: 25 - protein, 12 aa, Arabidopsis thaliana
DAGVIAGLNVAR

SEQ ID NO: 26 - protein, 11 aa, Arabidopsis thaliana
FDLTGVPPAPR

SEQ ID NO: 27 - protein, 10 aa, Arabidopsis thaliana
FEELNNDLFR

SEQ ID NO: 28 - protein, 7 aa, Arabidopsis thaliana
IMEYFIK

SEQ ID NO: 29 - protein, 8 aa, Arabidopsis thaliana
ILLESAIR

SEQ ID NO: 30 - protein, 12 aa, Arabidopsis thaliana
TSLAPGSGVVTK

SEQ ID NO: 31 - protein, 11 aa, Arabidopsis thaliana
FYSLPALNDPR

SEQ ID NO: 32 - protein, 14 aa, Arabidopsis thaliana
VVNFSFDGQPAELK

SEQ ID NO: 33 - protein, 15 aa, Arabidopsis thaliana
SENAVQANMELEFQR

SEQ ID NO: 34 - protein, 9 aa, Arabidopsis thaliana
VIAVGPGSR

SEQ ID NO: 35 - protein, 16 aa, Arabidopsis thaliana
EGDTVLLPEYGGTQVK

SEQ ID NO: 36 - protein, 11 aa, Arabidopsis thaliana
DEDVLGTLHED

SEQ ID NO: 37 - protein, 10 aa, Arabidopsis thaliana
TESGILLPEK

SEQ ID NO: 38 - protein, 16 aa, Arabidopsis thaliana
VIQPAKTESGILLPEK

SEQ ID NO: 39 - protein, 23 aa, Arabidopsis thaliana
LIPVSVKEGDTVLLPEYGGTQVK

SEQ ID NO: 40 - protein, 12 aa, Arabidopsis thaliana
TIAMDGTEGLVR

SEQ ID NO: 41 - protein, 10 aa, Arabidopsis thaliana
VVDLLAPYQR

SEQ ID NO: 42 - protein, 11 aa, Arabidopsis thaliana
IGLFGGAGVGK

SEQ ID NO: 43 - protein, 13 aa, Arabidopsis thaliana
VGLTGLTVAEYFR

SEQ ID NO: 44 - protein, 9 aa, Arabidopsis thaliana
FGLYYVDFK

SEQ ID NO: 45 - protein, 13 aa, Arabidopsis thaliana
EYADYVFTEYGGK

SEQ ID NO: 46 - protein, 7 aa, Arabidopsis thaliana
LSIAWSR

SEQ ID NO: 47 - protein, 16 aa, Arabidopsis thaliana
IGIAHSPAWFEPHDLK

SEQ ID NO: 48 - protein, 13 aa, Arabidopsis thaliana
EYADFVFQEYGGK

SEQ ID NO: 49 - protein, 13 aa, Arabidopsis thaliana
DFLSQGVRPSALK

SEQ ID NO: 50 - protein, 9 aa, Arabidopsis thaliana
FGLYYVDFK

SEQ ID NO: 51 - protein, 8 aa, Arabidopsis thaliana
NLNTDAFR

SEQ ID NO: 52 - protein, 14 aa, Arabidopsis thaliana
LDDIDFPEGPFGTK

SEQ ID NO: 53 - protein, 6 aa, Arabidopsis thaliana
SYYDKR

SEQ ID NO: 54 - protein, 8 aa, Arabidopsis thaliana
TLMNVFDK

SEQ ID NO: 55 - protein, 14 aa, Arabidopsis thaliana
TLMNVFDKTPNVDK

SEQ ID NO: 56 - protein, 23 aa, Arabidopsis thaliana
VFFSSSAVEYSNLAQAHATENAK

SEQ ID NO: 57 - protein, 16 aa, Arabidopsis thaliana
TVVDKSDDAPAETVLK

SEQ ID NO: 58 - protein, 16 aa, Arabidopsis thaliana
QYDGSDPQKPLLMAIK

SEQ ID NO: 59 - protein, 7 aa, Arabidopsis thaliana
FWDNFGK

SEQ ID NO: 60 - protein, 9 aa, Arabidopsis thaliana
FGWSANMER

SEQ ID NO: 61 - protein, 11 aa, Arabidopsis thaliana
YLSVTNPELSK

SEQ ID NO: 62 - protein, 12 aa, Arabidopsis thaliana
IYEMMDVALSGK

SEQ ID NO: 63 - protein, 12 aa, Arabidopsis thaliana
EVTTAEYNEFYR

SEQ ID NO: 64 - protein, 14 aa, Arabidopsis thaliana
AQSTGDTISLDYMK

SEQ ID NO: 65 - protein, 10 aa, Arabidopsis thaliana
IFGEDFLNDK

SEQ ID NO: 66 - protein, 9 aa, Arabidopsis thaliana
SIDSLVITK

SEQ ID NO: 67 - protein, 13 aa, Arabidopsis thaliana
YFFDGEIQSDKIK

SEQ ID NO: 68 - protein, 10 aa, Arabidopsis thaliana
VLTEFQEAAK

SEQ ID NO: 69 - protein, 11 aa, Arabidopsis thaliana
YFFDGEIQSDK

SEQ ID NO: 70 - protein, 14 aa, Arabidopsis thaliana
IDATEENELAQEYR

SEQ ID NO: 71 - protein, 17 aa, Arabidopsis thaliana
AEDDVNFYQTVNPDVAK

SEQ ID NO: 72 - protein, 18 aa, Arabidopsis thaliana
LAEGTDITSAAPGVSLQK

SEQ ID NO: 73 - protein, 12 aa, Arabidopsis thaliana
AVNVEEAPSDFK

SEQ ID NO: 74 - protein, 11 aa, Arabidopsis thaliana
WSAYVEDGKVK

SEQ ID NO: 75 - protein, 20 aa, Arabidopsis thaliana
SKLAEGTDITSAAPGVSLQK

SEQ ID NO: 76 - protein, 15 aa, Arabidopsis thaliana
NYINLAQIHASENSK

SEQ ID NO: 77 - protein, 9 aa, Arabidopsis thaliana
SFEQIEVER

SEQ ID NO: 78 - protein, 13 aa, Arabidopsis thaliana
INLAQIHASENSK

SEQ ID NO: 79 - protein, 10 aa, Arabidopsis thaliana
AIYTVGNWIR

SEQ ID NO: 80 - protein, 9 aa, Arabidopsis thaliana
IPTAELFAR

SEQ ID NO: 81 - protein, 10 aa, Arabidopsis thaliana
RIPTAELFAR

SEQ ID NO: 82 - protein, 19 aa, Arabidopsis thaliana
ALEEEIEDIGGHLNAYTSR

SEQ ID NO: 83 - protein, 10 aa, Arabidopsis thaliana
TILGPAQNVK

SEQ ID NO: 84 - protein, 25 aa, Arabidopsis thaliana
LSSDPTTTSQLVANEPASFTGSEVR

SEQ ID NO: 85 - protein, 11 aa, Arabidopsis thaliana
SASITGGYFYR

SEQ ID NO: 86 - protein, 9 aa, Homo sapiens
VSVGLLLVK

SEQ ID NO: 87 - protein, 10 aa, Homo sapiens
VFVAFQAFLR

SEQ ID NO: 88 - protein, 4 aa, Artificial Sequence
  peptide sequence tag
TTSF

SEQ ID NO: 89 - protein, 16 aa, Homo sapiens
YFSTTEDYDHEITGLR

SEQ ID NO: 90 - protein, 21 aa, Homo sapiens
LGALGGNTQEVTLQPGEYITK

SEQ ID NO: 91 - DNA, 1350 bp, Arabidopsis thaliana
tacgtataataggaaacaatcaacaaggaaattgaggagaaaaagacagaactccaaccaaaggtcgtggaaacctatgaagccacgtctgcagaagtcaaggtcgctatcatcttcttttttgttctccaagttacattaagtaattcaaatccatatttactgtaattaagactaaaattaaaacgagcaggctttggtgagagaccctaaggtggctggtttgaagaaaaactcagcggctgtgcagaagtacctcgaggagctagtcaaaattggtaaagattcaattacgctttgtcatgtgtctattttaaaatatcccgacaacaatatatgcttgtcttatatatttatcgttttttgtcttatatatcaaatatttgctggcaaaatttttggattcttatcatgtcctatattcttatacgcatgttatatctctagtttataagtcatgcatatatctttcatatagtataatcaaagttatttagcttgtatattggagtatttattatttttctatgaacatattatatcattctcattcaactagacgtaagtttcattcgtcatattcactcactttttgtttatggtctataatagtggttctctatggcttatttatttatatgatcttgcctaagaaaattggcacattcaaaaacctcaaactattgtggatcaaacttcatcatctttttcagaagacgtctagagattgtattggatcactgtttcttttgcatgatttataattagatgaacgttgtactcatctatgtctcaaattatgaaatgtttattcagaattccccggatcaaaagcggtgagtgaggcttcgtctagcttcggagctggctatgtcgcaggaccggtcacgttcatattcgagaaggtatctgttttcctcccggaggaggtgaagactaaagaaataccggtggaggaagtgaaagctgaagaacctgccaaaactgaagaaccagccaaaaccgaaggaacaagtggtgagaaagaggagattgttgaagagacaaagaaaggcgagacccctgaaaccgcggtcgtggaggagaagaaaccagaggtagaggagaagaaggaagaggctactccggctccggcagtggttgaaactccagttaaggaaccggagacaacgacgacagcgccagtggctgaaccaccaaagccttgatttgttttcaagatggtatgattacttctcttgtatgaaaacatctttgtacgtaacaaaaaaatgaaaggaagaaacggaaaggaacaaaaaaaaaaaacttatttttcattcttttttttcaatttggtttgtctttgtg

SEQ ID NO: 92 - protein, 9 aa, Arabidopsis thaliana
KYLEELVKI

SEQ ID NO: 93 - protein, 15 aa, Arabidopsis thaliana
KKGETPETAVVEEKK

SEQ ID NO: 94 - protein, 15 aa, Arabidopsis thaliana
KVVETYEATSAEVKV

SEQ ID NO: 95 - protein, 16 aa, Arabidopsis thaliana
KEIPVEEVKAEEPAKT

SEQ ID NO: 96 - protein, 18 aa, Arabidopsis thaliana
KTEGTSGEKEEIVEETKK

SEQ ID NO: 97 - protein, 22 aa, Arabidopsis thaliana
KKGETPETAVVEEKKPEVEEKK

SEQ ID NO: 98 - protein, 26 aa, Arabidopsis thaliana
KAVSEASSSFGAGYVAGPVTFIFEKV

SEQ ID NO: 99 - protein, 30 aa, Arabidopsis thaliana
KAEEPAKTEEPAKTEGTSGEKEEIVEETKK

SEQ ID NO: 100 - protein, 12 aa, Arabidopsis thaliana
KMKGRNGKEQKK

SEQ ID NO: 101 - protein, 238 aa, Artificial Sequence
  Genscan analysis
MGYWNSKVVPKFKKLFEKNSAKKAAAAEATKTFDESKETINKEIEEKKTELQPKVVETYEATSAEVKALVRDPKVAGLKKNSAAVQKYLEELVKIEVAIIFFFVLQVTLKFPGSKAVSEASSSGAGYVAGPVTFIFEKVSVFLPEEVKTKEIPVEEVKAEEPAKTEEPAKTEGTSGEKEEIVEETKKGETPETAVVEEKKPEVEEKKEEATPAPAVVETPVKEPETTTTAPVAEPPKP

SEQ ID NO: 102 - protein, 225 aa, Arabidopsis thaliana
MGYWNSKVVPKFKKLFEKNSAKKAAAAEATKTFDESKETINKEIEEKKTELQPKVVETYEATSAEVKALVRDPKVAGLKKNSAAVQKYLEELVKIEFPGSKAVSEASSSFGAGYVAGPVTFIFEKVSVFLPEEVKTKEIPVEEVKAEEPAKTEEPAKTEGTSGEKEEIVEETKKGETPETAVVEEKKPEVEEKKEEATPAPAVVETPVKEPETTTTAPVAEPPKP

SEQ ID NO: 103 - protein, 11 aa, Homo sapiens
KFLDQEEAEFL

SEQ ID NO: 104 - protein, 12 aa, Homo sapiens
RTVVMSSEAEIF

SEQ ID NO: 105 - DNA, 1500 bp, Homo sapiens
cctccaggagagccagggactcacccggcccttgtcccagactaactctggtcacagaaccatcctgtctgcctggaggggtggggtcccctgttctggcagaggtcacccccatatcaccgcatggggattttcttccctttgggtctctcttttcttcagagatgtatggccctggaggaggcaagtatttcagcaccactgaagactacgaccatgaaatcacagggctgcgggtgtctgtaggtcttctcctggtgaaaaggtgagtagggctatggtcatgggcccagcgccatgtcccctcccatcccacagtttcaggaactcagggcagggggtaagcacccgtggccacttttgccacacatgcctggctactgtcgatgcttcctggctcccgctgatgcttcctggctggagcggagacggtcagaccgtcctccctaccttctcccttcaacccaagctcaactcaaccaaaaatggcccctctgtccccatgcctgataggaaagtcaggggaaagtctgtccgattactgtcaaagaagacaggaggtaagggtcagagtggaccactgactgaatatgagtcgcagaagtgttagaggcagaagtccagggccatttccttaatatcgaagtgtctctgctggaggtctgggatggatttttgccctgcatttagaagttctggggtcctgggagaggggagagaagcccaatagcagaggagacagagtgtgggcggggcgagccggaggggtgcatcctgggagagcaccagggtgagggaggggtgaagatgagccccgtcagggaagcgctggcgagtgtgggaagtcacctgcccctcggcctgtgagctgctctgcttggagtgactaaggctcgggaggtccaggctcggccagaggcagctcatatgtgggccacagtgacggcagctggtgccttctgggtcacggagacctggcgctgcacgcagctctcctcaccaggatctcagtgactcctcccaaaagtcacacccactttgcagacggggaaactgagtccggagaggctgggtaacgagctcaagatcacagggcccaaaagtggtagaatcagggttggtgaccagtgagtctgtgtcagggacccaaagtctgatggtgctggactctctgcatcccgggaaggaggatgggggcgctgaggacccgggatgtgctgggccatcccagatctggacgtccaaagctttgcctctctcccagtgtccaggtgaaacttggagactcctgggacgtgaaactgggagccttaggtgggaatacccaggaagtcaccctgcagccaggcgaatacatcacaaaagtctttgtcgccttccaagctttcctccggggtatggtcatgtacaccagcaaggaccgctatttctattttgggaagcttgatggccagatctcctctgcctaccccagccaagaggggcaggtgctggtgggca

SEQ ID NO: 106 - protein, 8 aa, Homo sapiens
GMVMYTSK

SEQ ID NO: 107 - protein, 9 aa, Homo sapiens
VSVGLLLVK

SEQ ID NO: 108 - protein, 8 aa, Homo sapiens
DRYFYFGK

SEQ ID NO: 109 - protein, 10 aa, Homo sapiens
VFVAFQAFLR

SEQ ID NO: 110 - protein, 16 aa, Homo sapiens
YFSTTEDYDHEITGLR

SEQ ID NO: 111 - protein, 21 aa, Homo sapiens
LGALGGNTQEVTLQPGEYITK

SEQ ID NO: 112 - protein, 172 aa, Homo sapiens
MLLLLTLALLGGPTWAGKMYGPGGGKYFSTTEDYDHEITGLRVSVGLLLVKSVQVKLGDSWDVKLGALGGNTQEVTLQPGEYITKVFVAFQAFLRGMVMYTSKDRYFYFGKLDGQISSAYPSQEGQVLVGIYGQYQLLGIKSIGFEWNYPLEEPTTEPPVNLTYSANSPVGR

(19) United States
(12) Patent Application Publication
(10) Pub. No.: US 2003/0175722 A1
(43) Pub. Date: Sep. 18, 2003

(54) Methods and systems for searching genomic databases

(76) Inventors: Matthias Mann, Odense (DK); Peter Mortensen, Odense (DK)

Correspondence Address: ROPES & GRAY LLP, ONE INTERNATIONAL PLACE, BOSTON, MA, 02110-2624, US

(21) Appl. No.: 10/119,528

(22) Filed: Apr. 9, 2002

Related U.S. Application Data
(60) Provisional application No. 60/282,551, filed on Apr. 9, 2001.
(60) Provisional application No. 60/285,362, filed on Apr. 20, 2001.

(51) Int. Cl. C12Q 1/68; G01N 33/53; G06F 19/00; G01N 33/48; G01N 33/50

(52) U.S. Cl. 435/6; 702/20; 435/7.1; 702/19

(57) ABSTRACT

The instant invention provides methods and systems for searching genomic databases using polypeptide sequence information, such as those obtained from peptide sequencing projects, especially those using mass spectrometers. According to the instant invention, polypeptide sequences can be reverse translated into multiple sequence tags which are then used to search for identical or similar sequences in genomic databases, such as unanotated genomic databases of human or other organisms. Alternatively, the polypeptide sequences can be directly compared to sequences translated from at least 3, preferably all 6 reading frames of genomic sequences. The instant invention also provides systems for performing the methods of the instant invention, including computer systems, and systems including said computer systems and mass spectrometers linked to said computer systems. The instant invention further provides methods of conducting proteomic businesses using the methods of the instant invention.

### REFERENCES TO RELATED APPLICATIONS

This application claims priority to U.S. Provisional Application No. 60/282,551, filed on Apr. 9, 2001, and U.S. Provisional Application No. 60/285,362, filed on Apr. 20, 2001, the specifications of which are incorporated herein by reference in their entireties.

### BACKGROUND OF THE INVENTION

Systematic analysis of the function of genes can take place at the oligonucleotide or protein level. The latter has the advantage of being closest to function, since it is proteins that perform most of the reactions necessary for the cell. For most protein based (“proteomic”) approaches to gene function, mass spectrometry is the method of choice. Mass spectrometry can now identify proteins with very high sensitivity and medium to high throughput. New instrumentation for the analysis of the proteome has been developed including a MALDI hybrid quadrupole time of flight instrument which combines advantages of the mass finger printing and peptide sequencing methods for protein identification. New approaches include the isotopic labeling of proteins to obtain accurate quantitative data by mass spectrometry, methods to analyze peptides derived from crude protein mixtures and approaches to analyze large numbers of intact proteins by mass spectrometry directly.

To date, only protein sequence databases (usually auto-translated from DNA sequence data) and expressed sequence tag databases have been searched by mass spectrometric data. It has been shown that virtually all human proteins which can be visualized on gels can be identified in the expressed sequence tag databases, which now contain more than two million single read cDNA sequences. Neubauer et al. (1998) Nat. Genet. 20:46. However, ESTs usually cover only part of a protein sequence. If it were possible to work directly with genomic sequence databases, this would in principle allow for the identification of every peptide on which mass spectrometric sequence information was obtained. Difficulties in genome searching include the large size of the human genome and the fact that only a few percent are protein coding. Additionally, the exon-intron structure of most genes cannot be accurately predicted by bioinformatics. Krogh (1998) in: Guide to Human Genome Computing (Bishop, M. J., Ed.), pp. 26h1-274, Academic Press, San Diego; and Dunham et al. (1999) Nature 402:489.

### SUMMARY OF THE INVENTION

The present invention provides a method for identifying a coding sequence in a genomic database, e.g., an unannotated genomic database, comprising:

(i) generating, for an input polypeptide sequence, a set of sequence tags corresponding to possible coding sequences for the input polypeptide sequence; and

(ii) identifying, by an approximate string matching method using said sequence tags, genomic sequences from a genomic database which are similar to one or more of the sequence tags.

In certain preferred embodiments, the genomic database is an unannotated genomic database. In another preferred embodiment, the method also involves determining an open reading frame for the input polypeptide sequence in the genomic database, and, optionally, determining intron/exon boundaries in the open reading frame. In that manner, the subject method can be used to update, or provide a cross-referenced database, including coding sequence and intronic annotation for the genomic database.

In certain embodiments, the input polypeptide sequence is provided from a system for protein sequencing by mass spectrometry. For instance, the subject method can be performed by a computer which has a data link from a mass spectrometer system for transmitting the input polypeptide sequence.

In certain embodiments, the approximate string matching method is selected from the group consisting of a Shift-And method, a Karp-Rabin fingerprint method, and a Commentz-Walter method. In certain preferred embodiments, the approximate string matching method is a GREP method, such as an AGREP method. In general, the approximate string matching method will be one which tolerates a maximal number of errors, such as gaps for intronic sequence, of a size equal to at least the average length of intronic sequences in the genomic database.

In certain embodiments, the approximate string matching method has an error ratio, a, is less than 3.0, and even more preferably less than 1.0.

In certain embodiments, the subject method is carried out with multiple sequence tags, e.g., the multiple sequence tags are combined into a single array which is used as the input for the approximate string matching method.

Another aspect of the present invention provides a method for identifying a coding sequence in an unannotated genomic database, comprising:

(i) receiving an input polypeptide sequence; and

(ii) identifying, by an approximate string matching method using said input polypeptide sequence, coding sequences from a genomic database which has been dynamically translated in at least 3 reading frames.

Still another aspect of the present invention provides a computer system for identifying coding sequences in genomic databases, comprising:

(i) a sub-system for calculating and/or storing potential coding sequences for a polypeptide;

(ii) one or more databases of genomic sequence; and

(iii) an ID program for performing approximate string matching between nucleic acid sequences in a manner which accounts for differences between the two sequences due to an intronic sequence;

wherein, the system generates a set of sequence tags corresponding to possible coding sequences for an input polypeptide sequence, and identifies, from the database, any genomic sequences which are similar to one or more of the sequence tags, and indicates exon/intron boundaries, if any, in the genomic sequence(s).

For instance, in certain embodiments, the computer system also includes a sample/identification proteomics database for logging and correlating information such as sample identity, gel photos, mass spectra (and features therein), and search results.

The subject system can also include a transfer system to automate the transfer and utilization of mass spectrometric data of a target polypeptide.

Still another aspect of the present invention provides a mass spectrometry system including the above computer system and a mass spectrometer for sequencing polypeptides. For example, the spectrometer may include an ion source selected from the group consisting of electrospray and MALDI.

Yet another aspect of the present invention relates to a method of conducting a proteomics business, comprising:

(i) by the above-described method, determining the identity of a target gene encoding a protein isolated on the basis of the protein being (a) involved in an interaction of interest, (b) having a cellular localization of interest, (c) having a differential expression pattern of interest, or (d) being post-translationally modified;

(ii) identifying agents by their ability to alter the level of expression of the target gene or the activity of an expression product of the target gene;

(iii) conducting therapeutic profiling of agents identified in step (b), or further analogs thereof, for efficacy and toxicity in animals; and

(iv) formulating a pharmaceutical preparation including one or more agents identified in step (iii) as having an acceptable therapeutic profile.

The subject business method can include the additional step of establishing a distribution system for distributing the pharmaceutical preparation for sale, and may optionally include establishing a sales group for marketing the pharmaceutical preparation.

Still another aspect of the present invention provides a method of conducting a proteomics business, comprising:

(i) by the above-described method, determining the identity of a target gene encoding a protein isolated on the basis of the protein being (a) involved in an interaction of interest, (b) having a cellular localization of interest, (c) having a differential expression pattern of interest, or (d) being post-translationally modified;

(ii) (optionally) conducting therapeutic profiling of the target gene for efficacy and toxicity in animals; and

(iii) licensing, to a third party, the rights for further drug development of inhibitors or activators of the target gene.

### BRIEF DESCRIPTION OF THE DRAWINGS

FIG. 1: Scheme representing the steps which can be used to identify a gene locus from MS sequencing of a protein.

FIG. 2: Illustration of how MALDI sequence data can be used to extend exon coverage (SEQ ID NOS: 91-100).

FIG. 3: Comparison of performance of various sequence analysis algorithms with respect to predicting gene structure (SEQ ID NOS: 101-102).

FIG. 4: Two sequences retrieved from the human genome by the indicated peptide sequence tag. Correlation of calculated Y-ion series of the two sequences with the tandem MS spectrum reveals that only one sequence can be correct (SEQ ID NOS: 103-104).

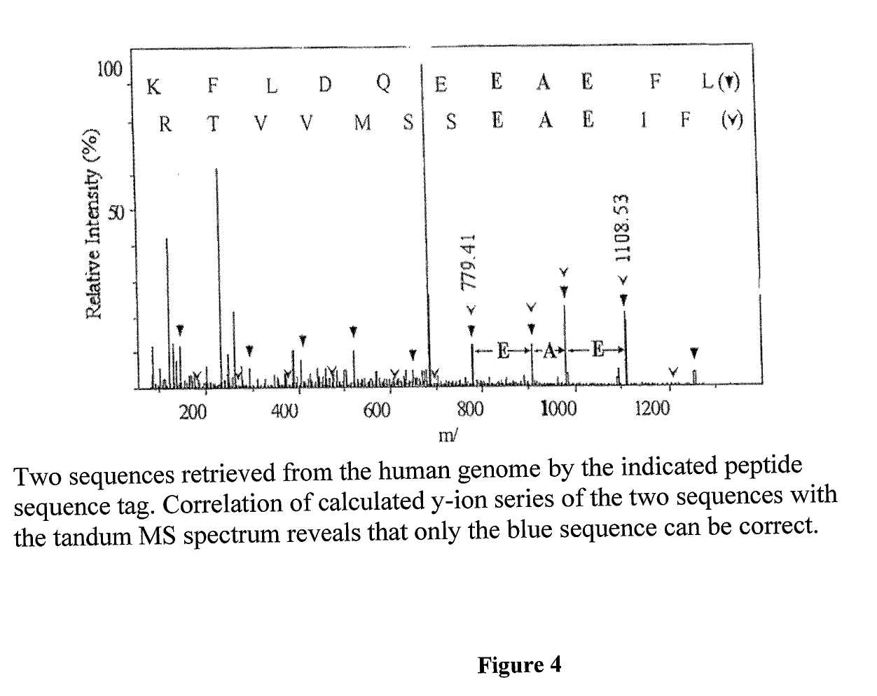

FIG. 5: Demonstrates the use of MS/MS and genome identification to elucidate the gene structure of a novel human protein (SEQ ID NOS: 105-112).

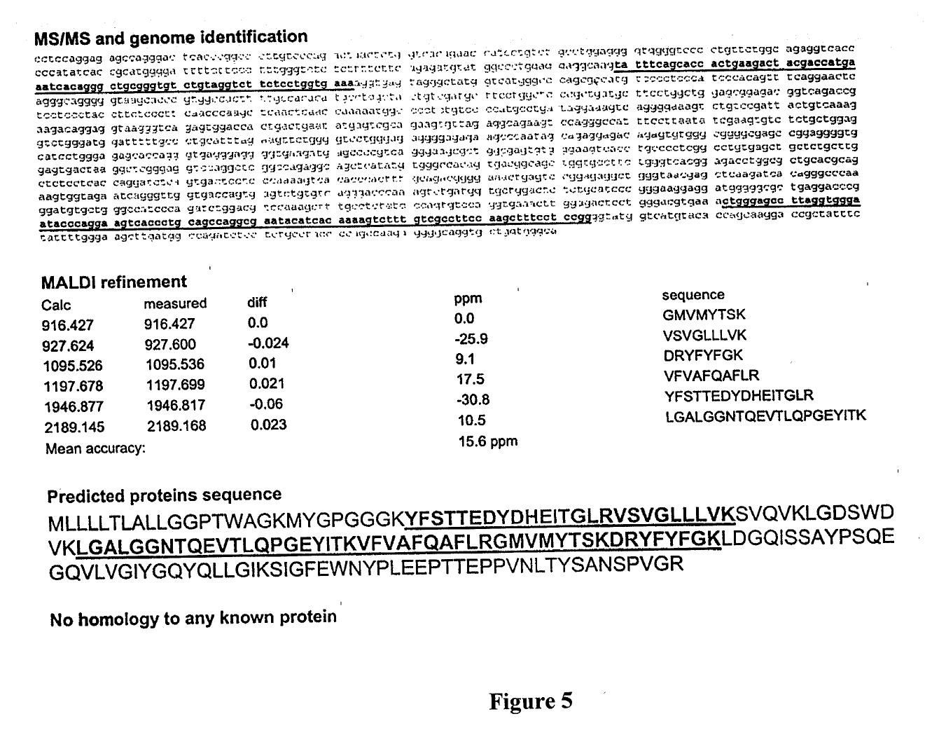

FIG. 6: Schematic representation of one preferred embodiment for information flow.

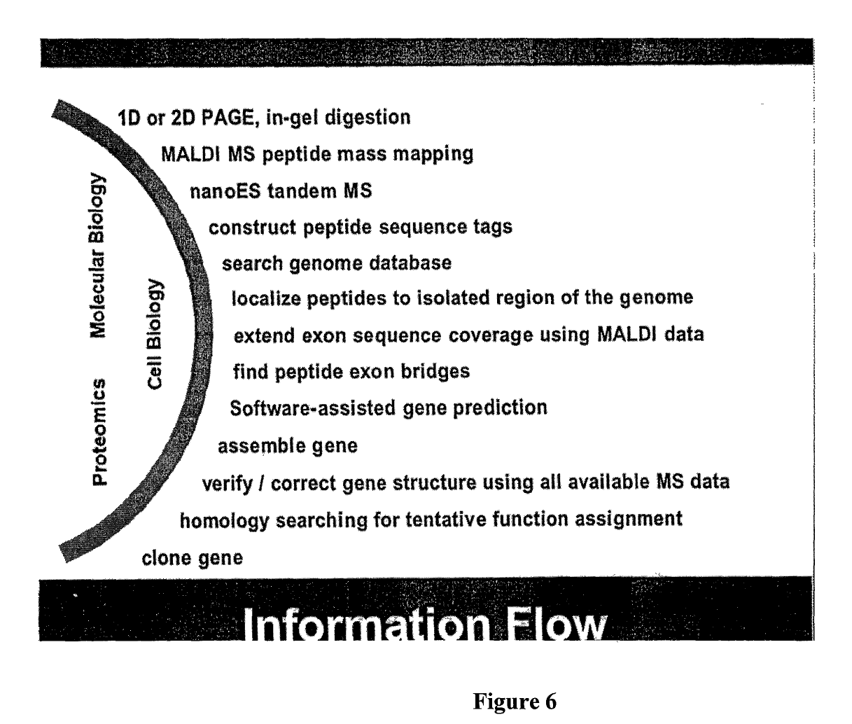

FIG. 7: Proposed information flow. All relevant information is stored in ProteomeDB, and unique Sample ID numbers are given. Links may also go directly to ProteomeDB from ProLogDB, ProAutoDB and the Prospects agents (not shown) in order to enter information without operator assistance.

FIG. 8: Main switchboard.

FIG. 9: Tables in ProteomeDB.

FIG. 10: Forms in ProteomeDB. In general, these parameters should not be modified unless by an administrator familiar with the database program such as MS Access.

FIG. 11: Reporting options in ProteomeDB. Reports can be transferred to a word processor (MS Word) by one button click and subsequently saved as a separate file (e.g. in rich text format) for easy distribution of analytical results via electronic means such as e-mail.

FIG. 12: ProteomeDB interface form.

FIG. 13: Search parameter window for peptide map queries.

FIG. 14: Search parameter window for peptide sequence tag queries.

FIG. 15: Search parameter window for breakpoint queries.

FIG. 16: Search parameter window for amino acid sequence queries.

FIG. 17: Sample information dialogue box.

FIG. 18: Search parameter window for automating ID program via ProAutoDB.

FIG. 19: Search parameter window for logging searches.

FIG. 20: Multi template interface.

FIG. 21: Search results window.

FIG. 22: 2nd pass check windows.

FIG. 23: Database entry window.

FIG. 24: Result summary window.

FIG. 25: ProLogDB browser window.

FIG. 26: Conversion of DNA sequence to amino acid sequence.

FIG. 27: Search parameter window for calculating theoretical fragment masses from a peptide sequence.

FIG. 28: Search parameter window for calculating theoretical peptide masses.

### DETAILED DESCRIPTION OF THE INVENTION

I. Overview

Large-scale DNA sequencing efforts are yielding the DNA blueprint of the human genome as well as of other organisms, and attention is now shifting to the systematic functional analysis of the biological information encoded by the genomes. Once aspect of these proteomics efforts utilizes mass spectrometry (MS) based protein identification, and relies on directly obtaining the sequence for a sample protein. While EST and other coding sequence libraries have been utilized to obtain identification of protein from a partial protein sequences, until the present invention, it had been unclear whether a genomic sequence information itself would be useful in the same way because of the vast size of genomes of higher organisms, the complex exon/intron structure of genes, and the large percentage of non-coding sequence. These features have made it very difficult to predict coding regions with certainty.

One aspect of the present invention is related to the demonstration that proteins can be directly sequenced using, e.g., mass spectrometry, and the coding sequences (along with information about intronic structures) for the proteins unambiguously identified in unannotated genomes as large as the human genome. A salient feature to the invention, as is described herein for embodiments utilizing mass spectrometry sequencing (“MS sequencing”), is that the subject method can be carried out using small amounts of proteins, e.g., sub-nanomol amounts of a test protein, and more preferably sub-picomol amounts of the test protein. In particular, the present invention is based on the discovery that a suitably modified pattern matching algorithm can be used with direct protein sequencing data to locate coding sequences in raw genomic data. In this regard, the mass spectrometric data can be used to predict the gene structure, such as intron/exon boundaries.

In one embodiment, the subject method is carried out as follows. Beginning with amino acid sequence data for a sample proteins, such as may be provided using mass spectrometry, a set of degenerate nucleotide sequences (“reverse transcribed sequences” or “sequence tags”) are calculated for the input polypeptide sequence. The set of sequence tags represents all, or at least the most likely based on codon usage, nucleotide sequences which could encode the sample protein. Utilizing each of the sequence tags, one or more similarity searches of a genomic database(s) is carried out in “forward” and “reverse” directions to identify similar sequence(s) in the genomic database. In certain instances, the subject method will utilize a pattern matching algorithm in the search which accounts for gaps in the similarity between the sequence tag and the genomic sequence, e.g., which accounts for and identifies the occurrence of intronic sequences which may disrupt the genomic coding sequence for the sample protein. This may be carried out utilizing further sequencing data, or by calculating intron/exon boundaries using known rules for intron splicing, and, for example, knowledge of the molecular weight of an unmodified form of the sample protein.

In other embodiments, the subject method is carried out by pattern searching with the amino acid sequence for the sample protein, against a set (e.g., six) of genomic sequence databases representing the genomic nucleotide sequence having been dynamically translated in all three reading frames. That is, the pattern matching is done at the level of actual amino acid sequence in a database of predicted amino acid sequences. As above, the subject method will preferably utilize a pattern matching algorithm which accounts for gaps in the similarity between the amino acid sequence of the sample protein and the dynamically translated genomic sequence in order to allow for intronic sequences which have been carried into the dynamically translated database in the form of non-sense amino acid sequence. For purposes of clarity, the application will now describe the subject method in terms of the use of sequence tags and genomic nucleotide sequence databases, though it will be understood that comparison at the level of amino acid sequence and dynamically translated genomic databases can be readily adapted for the various embodiments to be described.

In certain preferred embodiments, the subject method also utilizes homology searching to identify known, related proteins. Where only fragments of the sample protein have been sequenced, the sequence of identified homologs can be used to predict the remaining coding sequence and, accordingly, the intronic structure of the gene. The presence of homologs of known function can, of course, also provide guidance to the potential function of the sample protein.

The size of the human genome is approximately 25 times that of A. thaliana but the coding sequence is expected to be only 2-3 times larger. Tryptic peptides of the size typically encountered in MS sequencing (>10 aa) are almost always unique in the human genome. The information content of peptide sequence tags approximates that of the complete peptide sequence. In addition, the sequences retrieved by the search are checked against the tandem MS data which eliminates false positives. Therefore, searches using even short tags almost always result in unique identifications. Interestingly, the search specificity in the human genome is virtually identical to that of the dbEST but with the added advantage of high sequence accuracy, low redundancy and unbiased coverage.

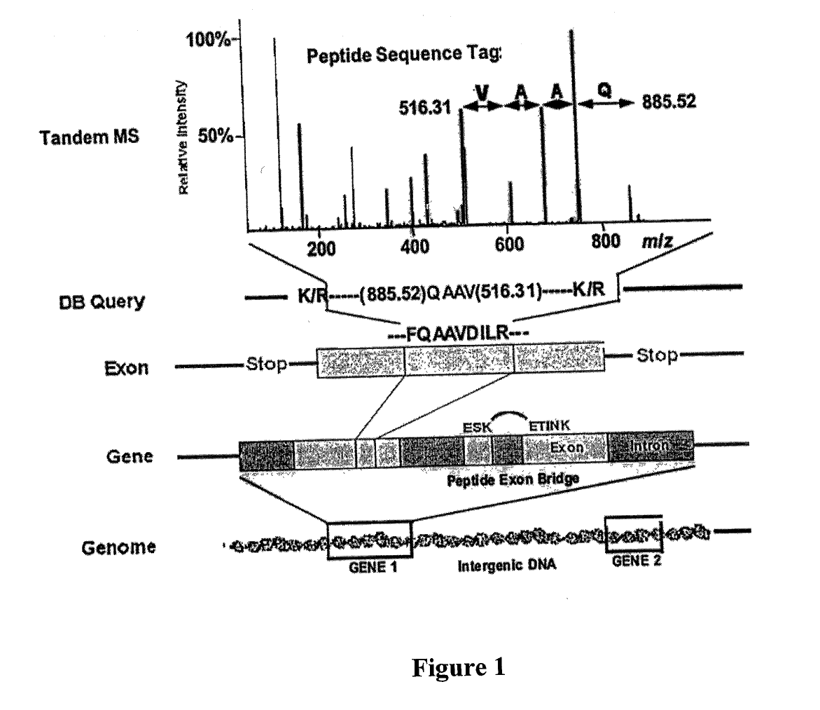

The following example illustrates the situation where peptide sequences correspond to coding regions within a gene. Referring to FIG. 1, whenever a peptide sequence tag derived from a MS/MS spectrum unambiguously identifies the corresponding DNA sequence in the genome, this sequence must be part of an exon. The peptide therefore locates the exon as well as the correct reading frame. In-frame stop codons upstream and downstream of the identified peptide also limit the extent of the exon within which the splice signals (exon intron boundaries) must be found. As described herein, Mass spectral data can be used to screen the vicinity of mapped regions for further exons. In many cases, peptides span two exons which enables the localization of the exact splice site for the two exons involved.

Typically, several peptides are partially sequenced during the course of a protein identification experiment using, for example, a mass spectrometer. Subsequent database searches identify peptides which cluster in a confined (2-15 kb) region of the genome which encompasses the underlying gene. The identified peptides define reading frames which in turn hold information about the intron/exon structure of the gene. Generally, two peptides are sufficient to identify and map the respective gene to its chromosomal location. Any of the identified exons can be used as probes for cloning or for homology searching for tentative function assignment. The defined genome area can be used to direct sequencing of further peptides in the same experiment.

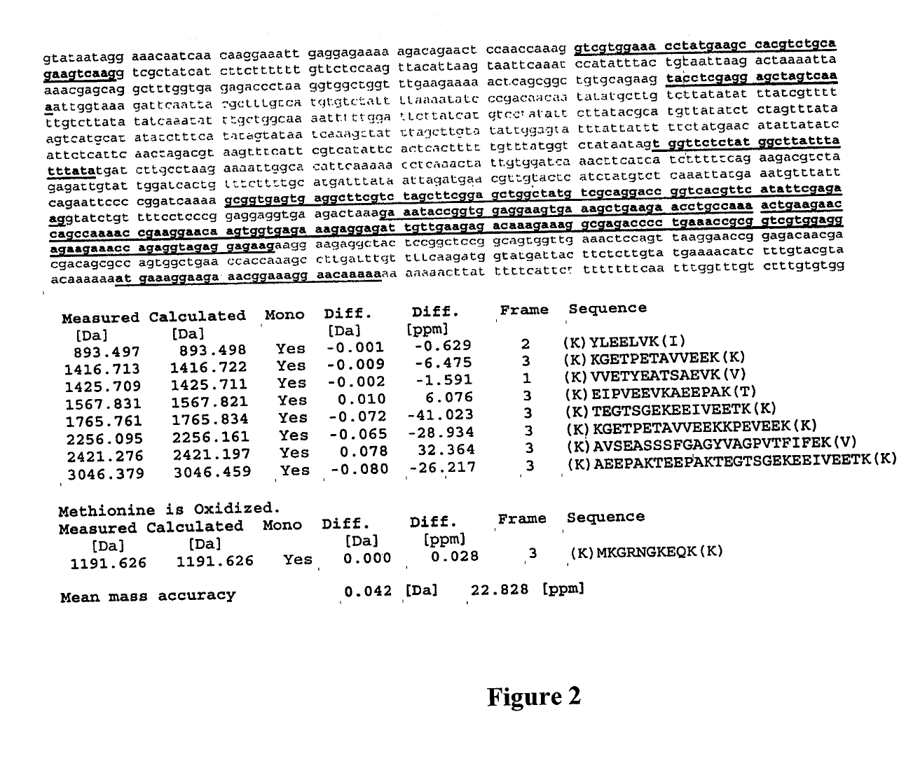

Most strategies for large scale protein identification follow a two tier analytical approach in which first a MALDI peptide mass fingerprint is created and samples that are not identified in this round of analysis are subjected to partial sequencing by tandem MS. It should be stressed that, owing to the complex structure of genes in higher organisms, fingerprint data alone does not hold sufficient discriminating power to identify proteins directly in a genome. As shown in FIG. 2, however, once part of the coding sequence of a protein has been found in the genome by peptide sequence tags, the 2-15 kb genomic sequence can be searched with the fingerprinting data by translating the nucleotide sequence in the three respective reading frames. Thereby, exon sequence coverage can be extended and additional exons can be found.

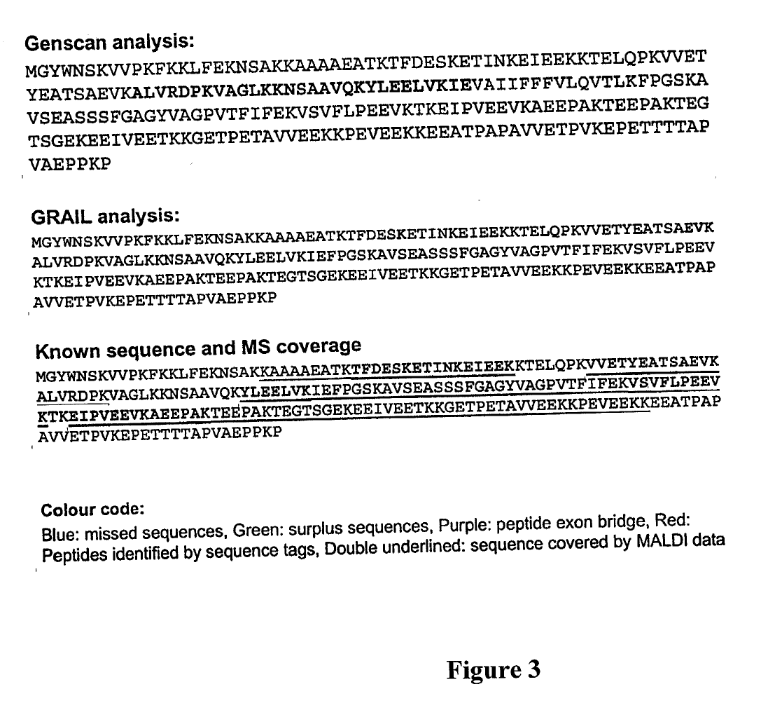

Computational gene prediction in genomic DNA of higher organisms has traditionally been very difficult but a combination of MS data and exon prediction can be very effective at defining gene structure. The genomic region identified in the previous figure was analyzed with GENSCAN, and GRAIL and compared to the known sequence of this protein. GENSCAN missed one exon and predicted a surplus one whereas GRAIL predicted two splice sites incorrectly. As shown in FIG. 3, MS data, in conjunction with the genome sequence, rectified the incorrect splice sites, led to inclusion of the exon that was missed and showed that the surplus exon was not present. The extent to which a predicted gene model can be verified or refined by the MS data obviously depends on the number of exons actually identified by peptide sequence tags.

II. Definitions

The term “percent identity” refers to the degree which residues in common at aligned positions between nucleic acid or amino acid sequences are said to be identical. For example, if they have 43 residues out of a total of 144 in common they are 29.9% identical.

The term “genomic information” includes protein coding regions, introns and other non-coding sequences, and other such structures that commonly appear genomic sequences. It is also meant to include the reading frame for proteins as encoded by a gene.

A “nucleotide residue” refers to-the nucleotide found along a polynucleotide sequence. For example, in a DNA sequence, it is mean to refer to adenine (A); guanine (G); cytosine (C); and thymine (T). For example, in a RNA sequence, it is mean to refer to adenine (A); guanine (G); cytosine (C); and uracil (U). This term can also include mutated and/or genetically engineered variations of nucleotide bases as are known in the art.

“ORF” or “Open Reading Frame” is a nucleotide sequence which could be translated into a polypeptide. Such a stretch of sequence is uninterrupted by a stop codon. An ORF that represents the coding sequence for a full protein begins with an ATG “start” codon and terminates with one of the three “stop” codons. For the purposes of this application, an ORF may be any part of a coding sequence, with or without start and/or stop codons. “ORF” and “CDS” may be used interchangeably.

The term “annotation” refers to the description of an ORF, introns and other genomic features.

A “contig” is a sequence derived by assembling two or more overlapping sequence fragments. For instance, a contig representing a portion of a CDS may be constructed by combining two or more overlapping EST sequences.

The term “allele” refers to alternative forms of a genetic locus; a single allele for each locus is inherited separately from each parent. The sequence of two alleles may identical or may different.

II. Methods for Pattern Matching

There are a variety of “pattern matching” or “approximate string matching” algorithms known in the art which can be readily adapted for use in the present invention. One problem in finding the coding sequence for a protein in genomic sequence databases can be formally stated as follows: given a genomic sequence of length n, a sequence tag of length m, and a maximal number of errors (e.g., gaps for intronic sequence) of k, find all segments of the genomic sequence (referred to herein as “occurrences” or “matches”) whose “edit distance” to the sequence tag is at most k. The edit distance between two sequences is defined as the minimum number of edit operations needed to transform one sequence into the other. The allowed edits in the context of the present invention include deleting, inserting and replacing nucleotide residues. The genomic sequence(s) and sequence tag(s) are sequences of characters from an alphabet Σ (of nucleotide residues) of σ. The error ratio, or error level, α can be given by α=k/m.

For instance, similarity tools developed by Needelman & Wunch (J. Mol. Biol. 48:444-453, 1970) and Sellers (SLAM J Appl Math. 26:787-793, 1974) can be used to calculate a global similarity score between the entire lengths of the sequences being compared. This type of algorithm is not sensitive for highly diverged sequences, but does not need to be so in most embodiments of the present method. Another available method focuses on shorter regions of local similarity. Examples of local similarity algorithms include the Smith-Waterman (J Mol Biol 147:195-197, 1981), BLAST (Altschul et al, J Mol Biol 215:403-410, 1990), and FASTA (Pearson and Lipman, PNAS 85:2444-2448, 1988).

In certain embodiments, the subject method uses a string matching method based on bit operations or on arithmetic, rather than character comparisons. Some of the examples are the Shift-And method, Karp-Rabin fingerprint method, or the algorithm of Commentz-Walter (“A string matching algorithm fast on the average” Proc. 6th International Colloquium on Automata, Languages, and Programming (1979), pp. 118-132), which combines the Boyer-Moore technique with the Aho algorithm.

In preferred embodiments, the subject method utilizes a pattern matching algorithm from the GREP family. One method for solving this problem is the algorithm described by Aho et al. (“Efficient string matching”, Communications of the ACM 18 (June 1975), pp. 333-340) which solves the problem in linear time. This algorithm is the basis of fgrep. As described in further detail, an exemplary embodiment of the method utilizes the AGREP algorithm, e.g., adapted from the teachings of Wu et al. (1992) Communications of the ACM, 35:83 and Wu et al. Proceedings of the Winter 1992 USENIX Conference San Francisco, 20-24. January 1992. pp. 153-162, Berkeley.

The AGREP algorithm is generally useful for problems where one is searching for a pattern P=p₁ p₂ . . . pₘ inside a large text file T=t₁ t₂ . . . tₙ. The pattern and the text are sequences of characters from a finite character set Σ. In certain embodiments of the subject method, the characters are DNA sequences, e.g., representing nucleotide bases, and are preferably genomic sequences. The AGREP method is used to find all occurrences of the reverse transcribed sequence (an “Sequence”) P in genomic sequence T; namely, it is used to search for the set of starting positions F={i|1≦i≦n−m+1 such that tᵢ tᵢ₊₁ . . . tᵢ₊ₘ₋₁=P}.

(A) Exact Match

In certain embodiments, the subject method uses an extract string matching method. To illustrate, let R be a bit array of size m (the size of the pattern). The term Rⱼ denotes the value of the array R after the j character of the nucleotide sequence has been processed. The array Rⱼ contains information about all matches of prefixes of P that end at j. More precisely, Rⱼ[i]=1 if the first i characters of the pattern match exactly the last i characters up to j in the sequence (i.e., p₁p₂ . . . pᵢ=tⱼ₋ᵢ₊₁tⱼ₋ᵢ₊₂ . . . tⱼ). For each read tⱼ₊₁ the method determines whether tⱼ₊₁ can extend any of the partial matches so far. For each i such that Rⱼ[i]=1, the system checks whether tⱼ₊₁ is equal to pᵢ₊₁. If Rⱼ[i]=0 then there is no match up to i and there cannot be a match up to i+1. If tⱼ₊₁=p₁ then Rⱼ₊₁ [1]=1. If Rⱼ₊₁[m]=1, then there is a complete match, starting at j−m+2, and the match is output, e.g., to a file, screen and or hardcopy. The transition from Rⱼ to Rⱼ₊₁ can be summarized as follows:

Initially, R₀[i]=0 for all i, 1≦i≦m; R₀[0]=1 (to avoid having a special case for i=1);

$${R}_{j + 1} [ i ] = \{ \begin{matrix}1 & if {R}_{j} [ i - 1 ] = 1 and {p}_{i} = {t}_{j + 1} \\ 0 & otherwise\end{matrix} ;$$

If Rⱼ₊₁[m]=1, then output a match at j−m+2.

The main observation about this transition, due to Baeza-Yates and Gonnet (in Proceedings of the 12th Annual ACM-SIGIR conference on Information Retrieval, Cambridge, Mass. (June 1989), pp. 168-175), is that it can be computed very fast in practice as follows. Let the “alphabet” be Σ=s₁, s₂, . . . , s_{|Σ|}. For each character sᵢ in the nucleotide sequence, one constructs a bit array Si of size m such that Si [r]=1 if pr=si. It is sufficient to construct the S arrays only for the characters that appear in the pattern. In other words, Sᵢ denotes the indices in the pattern that contain si. It is easy to verify now that the transition from Rⱼ to Rⱼ₊₁ amounts to no more than a right shift of Rⱼ and an AND operation with Sᵢ, where sᵢ=tⱼ₊₁. So, each transition can be executed with only two simple arithmetic operations, a shift and an AND. Assume that the right shift fills the first position with a 1. If only 0-filled shifts are available (as is the case with C), then the system can add one more OR operation with a mask that has one bit. (Baeza-Yates and Gonnet, supra, used 0 to indicate a match and an OR operation instead of an AND; that way, 0-filled shifts are sufficient.

(B) Matching With Errors

In more preferred embodiments, however, the subject method utilizes an algorithm which tolerates errors (mismatches), e.g., for approximate pattern matching between the sequence tag and genomic sequence(s). In one embodiment, the previously described method can be adapted to allow errors in matching. As a simple illustration, the method can be adapted to permit one insertion into the pattern at any position. In other words, the method finds all intervals of size at most m+1 in the genomic sequence that contain the pattern of the sequence tag as a subsequence. The R and S arrays are defined as before, but now there are two possibilities for each prefix match. There can be an exact match or a match with one insertion. Accordingly, another array is introduced, denoted by Rⱼ¹ which indicates all possible matches up to tⱼ with at most one insertion. More precisely, Rⱼ¹[i]=1 if the first i nucleotides of the pattern match i of the last i+1 characters up to j in the sequence. If both R and R¹ are maintained, then all matches can be found which have at most one insertion: Rⱼ[m]=1 indicates that there is an exact match and Rⱼ¹[i]=1 indicates that there is a match with at most one insertion (sometimes both will equal to 1 at the same time).

The transition for the R array is the same as before. One need only to specify the transition for R¹. There are two cases for a match with at most one insertion of the first i characters of P up to tⱼ₊₁:

I1. There is an exact match of the first i nucleotides up to tⱼ. In this case, inserting tⱼ₊₁ at the end of the exact match creates a match with one insertion.

I2. There is a match of the first i−1 nucleotides up to tⱼ with one insertion and tⱼ₊₁=pᵢ. In this case, the insertion is somewhere inside the sequences and not at the end.

Case I1 can be handled by just copying the value of R to R¹, and case I2 can be handled with a right shift of R¹ and an AND operation with Sᵢ such that sᵢ=tⱼ₊₁. So, to compute Rⱼ¹, one additional shift (the shift of R is done already) is done, one AND operation and one OR operation.

In another embodiment, the method can allow for one deletion between the sequences (and no insertions). R, R¹ (which now indicates one deletion), and S are as defined before. There are again two cases for a match with at most one deletion of the first i characters of P up to tⱼ₊₁:

D1. There is an exact match of the first i−1 characters up to tⱼ₊₁ (which is indicated by the new value of the R array Rⱼ₊₁ [i−1]). This case corresponds to deleting pi and matching the first i−1 characters.

D2. There is a match of the first i−1 characters up to tj with one deletion and tj+1=pi. In this case, the deletion is somewhere inside the sequence and not at the end. Case D2 is handled as before (it is exactly the same), and case D1 is handled by a right shift of the new value of Rj+1.

In still another embodiments, the method can allow for a substitution. That is, it allows for replacing one nucleotide of P with one nucleotide of T. Again, there are two cases:

S1. There is an exact match of the first i-1 nucleotides up to tⱼ. This case corresponds to substituting tⱼ₊₁ with pᵢ (whether or not they are equal—the equality will be indicated in R) and matching the first i−1 nucleotides.

S2. There is a match of the first i-1 nucleotides up to tⱼ with one substitution and tⱼ₊₁=pᵢ. In this case, the substitution is somewhere inside the sequence and not at the end

Case S2 is again the same. Case S1 corresponds to looking at Rⱼ[i−1] as opposed to looking at Rⱼ₊₁[i−1] in case D1.

However, in certain preferred embodiments, the subject method handles the general case of up to k errors, where an error can be either an insertion, a deletion, or a substitution (the Levenshtein or the edit-distance measure). Overall, instead of one additional R¹ array, k additional arrays R¹, R², . . . , R^{k} are maintained, such that array R^{d} stores all possible matches with up to d errors. The transition from array Rⱼ^{d} to Rⱼ₊₁^{d} is determined. There are 4 possibilities for obtaining a match of the first i nucleotides with ≦d errors up to tⱼ₊₁:

1. There is a match of the first i−1 nucleotides with ≦d errors up to tⱼ and tⱼ₊₁=pi. This case corresponds to matching tⱼ₊₁.

2. There is a match of the first i−1 nucleotides with ≦d−1 errors up to tⱼ. This case corresponds to substituting tⱼ₊₁.

3. There is a match of the first i−1 nucleotides with ≦d−1 errors up to tⱼ₊₁. This case corresponds to deleting pᵢ.

4. There is a match of the first i nucleotides with ≦d−1 errors up to tⱼ. This case corresponds to inserting tⱼ₊₁.

R is denoted as R⁰, and the method assumes that t^{j+1}=s_{c}. The subject method can provide the following expression for Rⱼ₊₁^{d}:

R₀^{d}=11 . . . 100 . . . 000 d ones.

$$\begin{matrix}{R}_{j + 1}^{d} = Rshift [ {R}_{j}^{d} ] AND {S}_{c} OR Rshift [ {R}_{j}^{d - 1} ] OR Rshift [ {R}_{j + 1}^{d - 1} ] OR {R}_{j}^{d - 1} \\ = Rshift [ {R}_{j}^{d} ] AND {S}_{c} OR Rshift [ {R}_{j}^{d - 1} OR {R}_{j + 1}^{d - 1} ] OR {R}_{j}^{d - 1} .\end{matrix}$$

Overall, there are total of two shifts, one AND, and three ORs for each R_{d}. There are k+1 arrays, so the total amount of work is O((k+1)n). An important feature of this algorithm is that it can be relatively easily extended to several more complicated patterns, including accounting for intronic sequences present in the genomic sequence.

If the number of errors is small compared to the size of the pattern, then the running time can be improved in some instances by what is referred to as the partition approach. Suppose again that the pattern P is of size m and that at most k errors are allowed. Let

$$r = ⌊ \frac{m}{k + 1} ⌋ ,$$

and let P₁, P₂, . . . , Pₖ₊₁ be the first k+1 blocks of P each of size r. In other words, P₁=p₁ p₂ . . . pᵣ, . . . , Pⱼ=p₍ⱼ₋₁₎ᵣ₊₁ . . . pⱼᵣ. If P matches the text with at most k errors, then at least one of the Pⱼ's must match the sequence exactly. All Pⱼ's can be searched at the same time and, if one matches, then the whole pattern can be checked directly within a neighborhood of size m from the position of the match. Since, in this embodiment, the method looks for an exact match, there is no need to maintain all k of the R^{d} vectors. This scheme will run fast if the number of exact matches to any one of the Pⱼ's is not too high. The number of such matches depend on many factors including the values of r and m.

(C) Multiple Patterns

In the process of calculating the various potential coding sequences for a given amino acid sequence, the subject method will generating multiple sequence tags. In general, one will want to find all occurrences of any of these sequence tags. Under those circumstances, the pattern searching against the genomic sequence(s) can be conducted one at a time or together.

The advantage of searching for all of sequence tags together is that it can be done in one scan (and in one command). Suppose that one is looking for P₁, P₂, . . . , Pᵣ. All of the sequence tags can be concatenated and put in one array (using as many words as needed), and apply the algorithm on that array with the following modifications. Let M be a bit array the size of the combined sequence tags, and let bit i be 1 if and only if i corresponds to the first character of any of the sequence tags. For each sεS, two bit arrays are built. The first, Sₛ is identical with the one described above. It is used to determine if a match occurs. The second array S′ₛ=Sₛ AND M. It indicates whether s is the first character of any pattern. If so, then one must start the match at that pattern: e.g., the method should not depend on the end of the previous pattern. Thus, after computing Rⱼ, the method performs an OR function with S′ₛ (where s=tⱼ). The rest of the R arrays are computed as before, except that in each step they are OR'd to a special mask that sets the first d bits in R^{d} of each separate pattern to 1; this allows d initial errors in each pattern.

The multi-pattern matching algorithm described above can be used to solve the approximate string-matching problem for searching reverse translated sequences against genomic sequences. Let P=p1, p2, . . . , pM be a pattern string, and let T=a1, a2, . . . aN be a text string. We partition P into k+1 fragments P1, P2, . . . , Pk+1, each of size m=M/(k+1). Let Tij=ai, . . . , aj be a substring of T. By a pigeonhole principle, if Tij differs from P by no more than k errors, then one of the fragment must match a substring of Tij exactly.

The approximate string matching algorithm is conducted in two phases. In the first phase the sequence is partitioned into k+1 fragments and uses the multi-pattern string matching algorithm to find all those places in the genomic sequence that contain one of the fragments. If there is a match of a fragment at position i of the genomic sequence, the system marks the positions i−M−k to i+M+k−m as a “candidate” area. After the first phase is done, an approximate matching algorithm as described above to find the actual matches in those marked area. In an illustrative embodiment, the pseudo-code for the subject method may be illustrated by:

Let p be the current position of the text ;
while (p < N) /* N is the end position of the sequence text */
{

blk_idx = map(ap −b +1 ap −b +2 . . . ap ) /* map transforms
a string of size b into an integer */
shift_value = SHIFT [blk_idx ];
if (shift_value > 0) p = p + shift_value;
else

compute the hash value of ap −μ+1 . . . ap ;
compare ap −μ+1 . . . ap to every pattern that has
the same hash value;
if there is a match then reports ap −μ+1 . . . ap ;
p = p + 1;

}

IV. Uses in Proteomics

Mass spectrometry has emerged as a central technique in a wide variety of functional genomics, or proteomics approaches to study gene function in the post-genomics world. Mass spectrometric instrumentation continues to become more powerful and novel instrumental concepts are being put into use. The subject genomic searching system can be used as part of a proteomics discovery method.

For instance, the subject method can use peptide sequence information obtained by mass spectrometry as the identification method in “expression proteomics”, sequencing data from with two-dimensional gels of two different biological states.

Several interesting approaches have been taken recently towards the analysis of the proteome without the use of gel electrophoresis. In one such approach, the protein population is separated by a variant of capillary electrophoresis and the intact proteins are then eluted into a Fourier transform ion cyclotron resonance mass spectrometer (FT ICR). The FT ICR is capable of storing the ions and measuring them at extremely high resolution and mass accuracy using a frequency based method. Measurement of several hundreds protein components from lysates of Escherichia coli or yeast has already been shown. Jensen et al. (1999) Anal. Chem. 71:2076. Using a variant of the tandem mass spectrometric method, it may also be possible to identify the proteins “on-line” as they elute into the mass spectrometer. See, for example, Mørtz et al. (1996) PNAS 93:8264-8267; and Li et al. (1999) Anal. Chem. 71:4397.

In another approach, crude protein mixtures are digested in solution without separation. The resulting peptide mixture is then analyzed by the LC/MS method outlined above. Yates et al. (1997) Protein Chem. 16:495; and Link et al. (1999) Nat. Biotechnol. 17:676. As the capacity of the mass spectrometer to sequence co-eluting peptides increases, more and more complex protein mixtures can be analyzed.

(A) Multi-Protein Complexes

In one embodiment, the subject method is used to search genomic databases for sequences derived from multi-protein complexes, e.g., assemblies with a particular function such as splicing, transport or nuclear import/export. One use of proteomics technology is to determine the make up of such complexes. To this end, they need to be purified specifically, the identity of the factors in the complex needs to be determined and finally the in vivo presence of the novel members of the complex needs to be established.

(B) Signaling Pathways

The subject method can also be used as part of a proteomic discovery method to elucidate transient rather than structural complexes. Many signaling cascades are transmitted through multi-protein complexes involving scaffolds and these complexes can be biochemically purified.

(C) Organelles

In still other embodiments, e.g., apart from multi-protein complexes, the subject method can be used identify proteins in cellular organelles. For instance, organelles can be purified and their composition analyzed by mass spectrometry. Since organelles are often less well defined than smaller multi-protein complexes, the task of verification of identifications becomes even more important.

V. Business Methods

Yet another aspect of the present invention relates to a method of conducting a proteomics business, comprising:

(i) by the above-described method, determining the identity of a target gene encoding a protein isolated on the basis of the protein being (a) involved in an interaction of interest, (b) having a cellular localization of interest, (c) having a differential expression pattern of interest, or (d) being post-translationally modified;

(ii) identifying agents by their ability to alter the level of expression of the target gene or the activity of an expression product of the target gene;

(iii) conducting therapeutic profiling of agents identified in step (b), or further analogs thereof, for efficacy and toxicity in animals; and

(iv) formulating a pharmaceutical preparation including one or more agents identified in step (iii) as having an acceptable therapeutic profile.

The subject business method can include the additional step of establishing a distribution system for distributing the pharmaceutical preparation for sale, and may optionally include establishing a sales group for marketing the pharmaceutical preparation.

Still another aspect of the present invention provides a method of conducting a proteomics business, comprising:

(i) by the above-described method, determining the identity of a target gene encoding a protein isolated on the basis of the protein being (a) involved in an interaction of interest, (b) having a cellular localization of interest, (c) having a differential expression pattern of interest, or (d) being post-translationally modified;

(ii) (optionally) conducting therapeutic profiling of the target gene for efficacy and toxicity in animals; and

(iii) licensing, to a third party, the rights for further drug development of inhibitors or activators of the target gene.

VI. Exemplary System

In an illustrative embodiment, there is provided a protein identification program (ID program) comprising two main components: a server application with sequence database search routines that include client interface(s). Merely to illustrate, the ID program can be automated via the Microsoft Access databases ProAutoDB and ProLogDB and associated Visual Basic applications. Control of automation and data flow can be as follows: from the ID program GUI it is specified to query e.g. the ‘FavoritelndexFile’ from a list of several virtual index files. Elsewhere it is specified that ‘FavoritelndexFile’ is actually e.g. particular genomic sequence databases. Upon finding matches with scores higher than a predefined value, the search result and all search parameters can be logged, also in another prespecified database, and further searches on the dataset can be aborted or continued as predefined in the automation database.

Special automated actions can also be triggered by certain database retrieval events, e.g. the matching of a data set to a specific ORF (Open Reading Frame) could result in an e-mail being sent with all available information to a person with particular interest in this gene/protein.

A few terms used in these examples include:

“ProteomeDB”: A sample/identification proteomics database for logging and correlating information such as sample identity, gel photos, mass spectra (and features therein), search results, etc. This database can be the final destination of data but can also be regarded as a temporary storage facility for data that is subsequently transferred by, e.g., standard SQL commands to other databases (e.g. Oracle and Sybase databases) on a remote server.

“ID program Flow Agent”: A software daemon to automate the transfer and utilization of mass spectrometric data.

“ProAutoDB”: Database(s) containing search parameters and related information regarding data sets that have been scheduled for later automated (and repeated) searching against sequence databases.

In certain embodiments, all incoming samples are logged into ProteomeDB, before any analyses are carried out. Each logged sample is then automatically given a unique ID number that can be used to sort subsequently generated mass spectrometric data and database search results. In certain preferred embodiments, ProteomeDB will be able to download digestion protocols to a robotic workstation and supply all relevant sample information directly into MALDI and ES mass spectrometer control software. This means that setting up the analysis of a batch of samples will be done automatically.

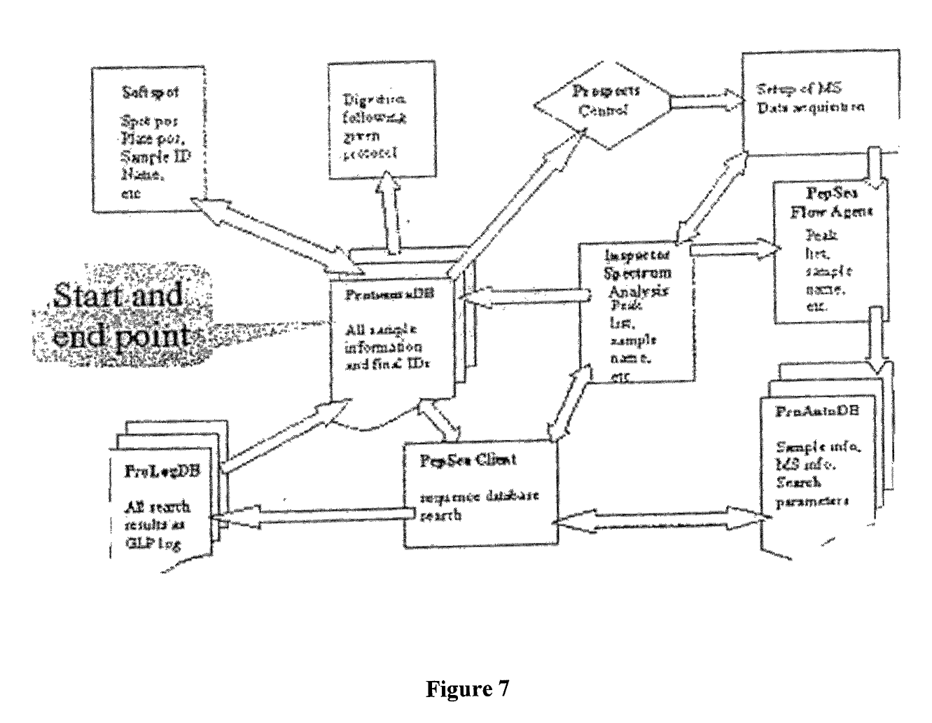

Referring to FIG. 7, mass spectrometric data is acquired either: i) manually; ii) automatically through built in features in the MS software; or iii) governed by scripts. The relevant MS information, e.g. a peptide mass list or fragment mass list is passed to ProAutoDB either by the MS control software directly, or via ID program Flow Agent. ID program Client checks ProAutoDB for new tasks at set intervals and upon finding a job then executes the sequence database search. The outcome of every search is logged in ProLogDB, and if sequence database entries achieve a scoring value above a set threshold then these proteins are also logged back into ProteomeDB under the pertinent sample record.

(i) ProteomeDB Files

In the illustrated example, ProteomeDB is a hierarchic database as can be developed for Microsoft Access. It may contain tables, forms, reports and a VisualBasic module or the like. Briefly, a batch can have many samples, each of which can have many mass spectra, each of which can have many database search results. The form set and the database tables can also be separated (called ‘split’) such that the data can reside on a central file server and be simultaneously accessible to a group of users, each of whom should have a copy of the form set on their computer.

(ii) The Navigation Switchboards

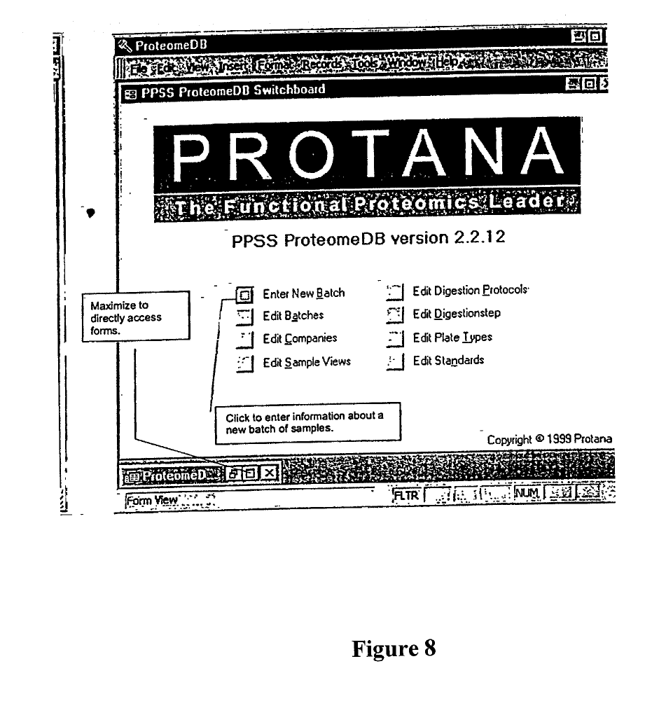

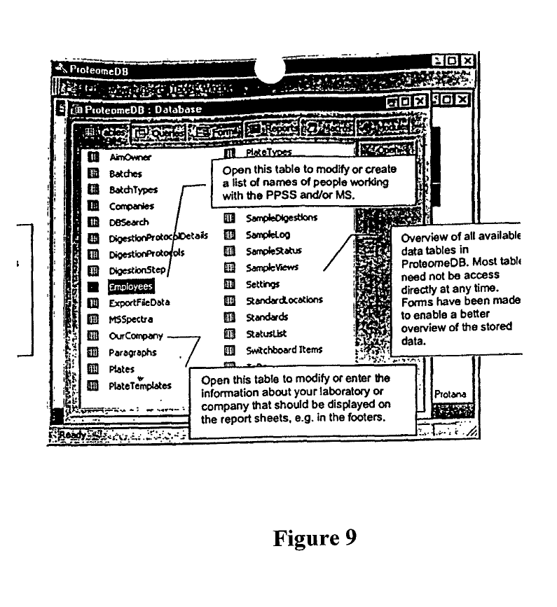

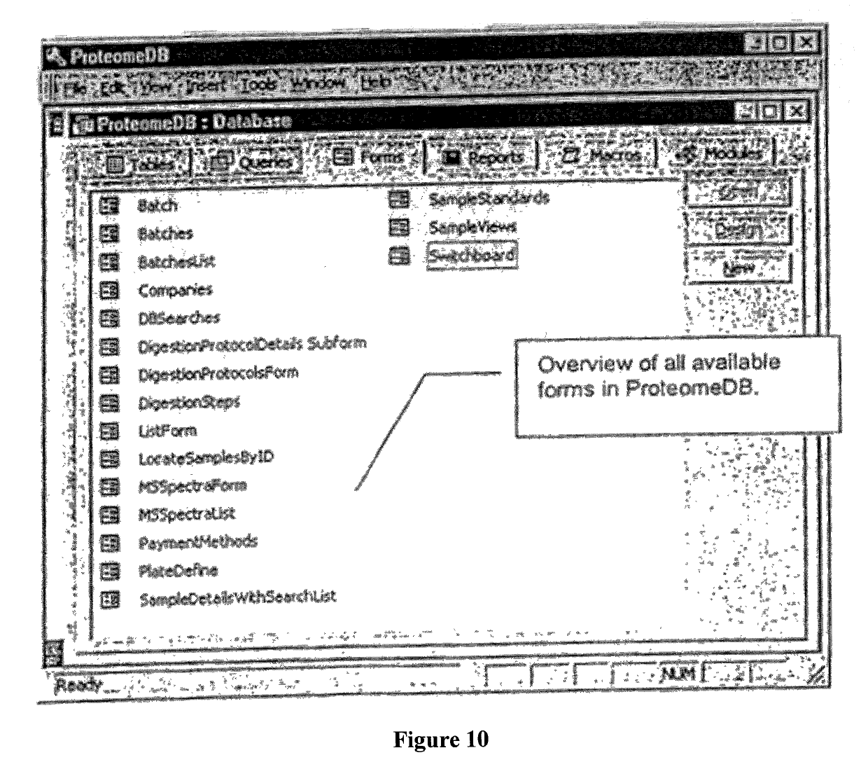

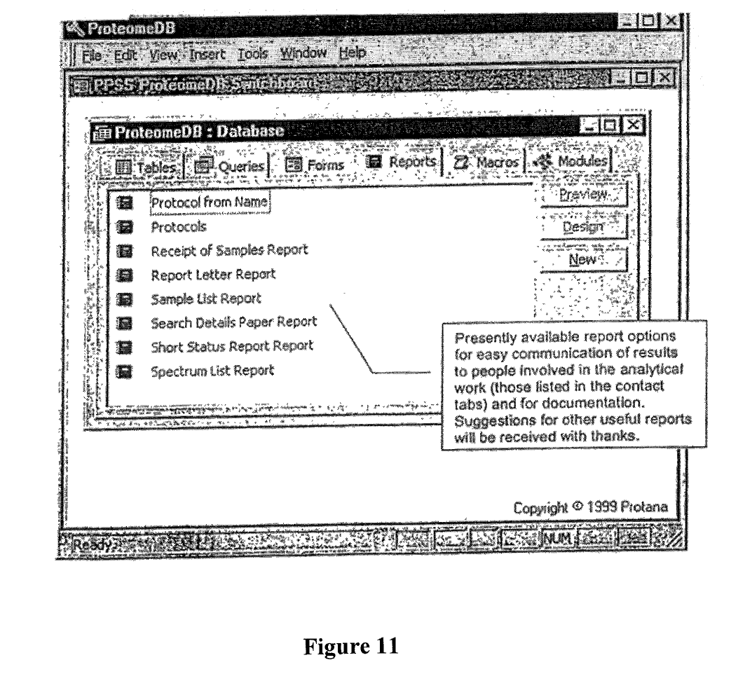

FIG. 8 shows an exemplary first window that becomes available after opening ProteomeDB is the main ‘Switchboard’. The appearance of the switchboard can be modified to display the logo and colors of a company. The ‘Enter New Batch’ button can be used to enter the data relating to a new batch of samples. One or more secondary switchboards give access to most of the sub-forms for more direct and simplified entry of data (e.g., going into one table at a time). See FIGS. 9-11.

(iii) Batch Overview: Information Relating to the Entire Batch

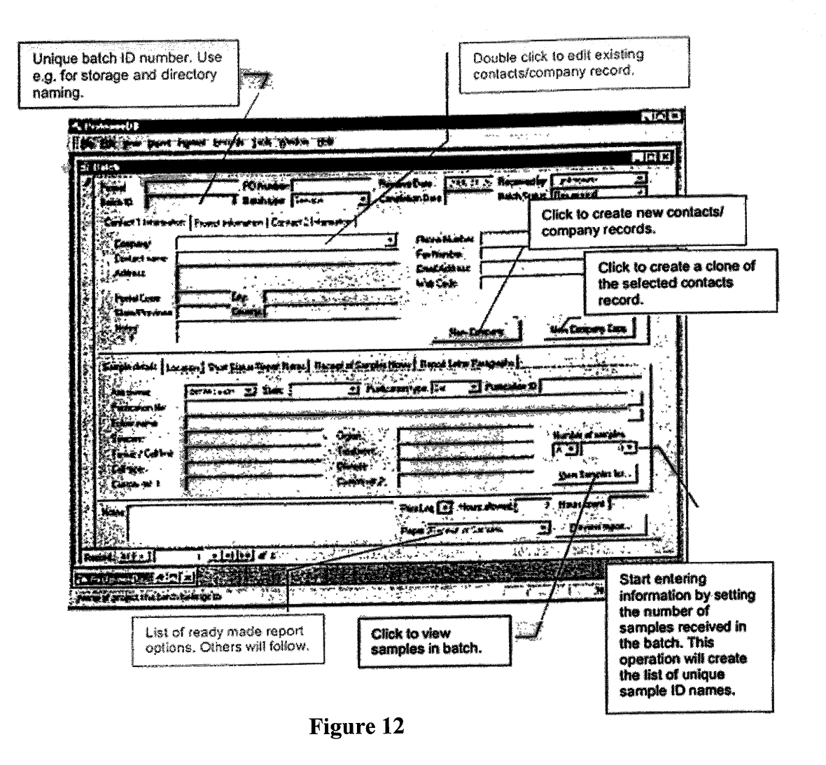

The primary batch information can be one record. See FIG. 12. These two forms, or views, can be set up to be the most used ProteomeDB interfaces; i.e., they are the ‘top level’ where a batch of samples is set in line for analysis and the report option is finally chosen. Ideally, it is only necessary to type in the number of samples in the batch and the name given to each sample by the owner of the batch. There is no way of predicting what Web call their samples, so this task is preferably not automated. The information in all the sub forms and surrounding bits of information may be either:

entered automatically at different stages by other applications. Examples of this functionality are spectrum names, peak lists, analysis dates etc.

reused information from earlier batches. Examples of reuse are digestion protocols (and protocol steps), contact person information, etc.

(iv) Companies Sub Form

The Companies sub form shows all the information stored on each single contact person. Not all information is necessarily used for each role that the contact subsequently has. For example, only the person chosen in the ‘Contacts information’ tab may be allocated the Web access code, and only the person in the tab ‘Billing information’ shows as having a Tax identification number associated.

List of Samples: A list can be provided which contains the information for each batch, namely the sample names along with the corresponding identification or sequence information that was found. When setting up a new batch, the Web can be prompted to start by setting the number of samples in order to obtain an auto-generated list of unique sample ID numbers. The analysis status of a new sample can be by default ‘Received for identification’, with other status possibility chosen manually, such as for example, ‘Received for sequencing’.

Prewritten report options: In certain embodiments, the ProteomeDB can maintain reports, e.g., for printing or electronic documentation, in separate text files pertaining to the relevant analytical results from each batch. To illustrate, the system may offer some report options that exclusively deal with the results:

The ‘Short status report’: a very short information abstract that shows the results from the entire batch on one or a few pages.

The ‘Search Details paper’: includes results lists from each search and can occupy several pages per sample. In many cases, the researchers doing the actual protein analysis may not be the ones who own (submit) the samples and need the results. This means that communication of results to other parties is needed, and for that purpose there are some extra report options;.

The ‘Receipt of samples’ fax: to confirm the arrival of the samples, also states the batch ID and Web access code that will allow the owner of the samples to follow the analysis progress via the Web.

The ‘Report letter’: a letter to accompany one or all of the result reports mentioned above. The information that needs to be conveyed about the analyses can often be similar from project to project. Therefore it may be useful to have a selection of informative standard paragraphs that may be included (or excluded) in this letter.

The ‘Invoice’: This module may generate the finished invoice, or can be used for interdepartmental billing. Invoice numbers can be assigned when the ‘Batch status’ is changed to ‘Completed’.

Information about the batch: Date fields allow entry of the dates where: I) the samples were dispatched by their owner; II) the samples were received for analysis; and III) the analyses were completed. In certain embodiments, the system will provide queries to check current status at various stages of the project work.

(v) User Designated Search Parameters

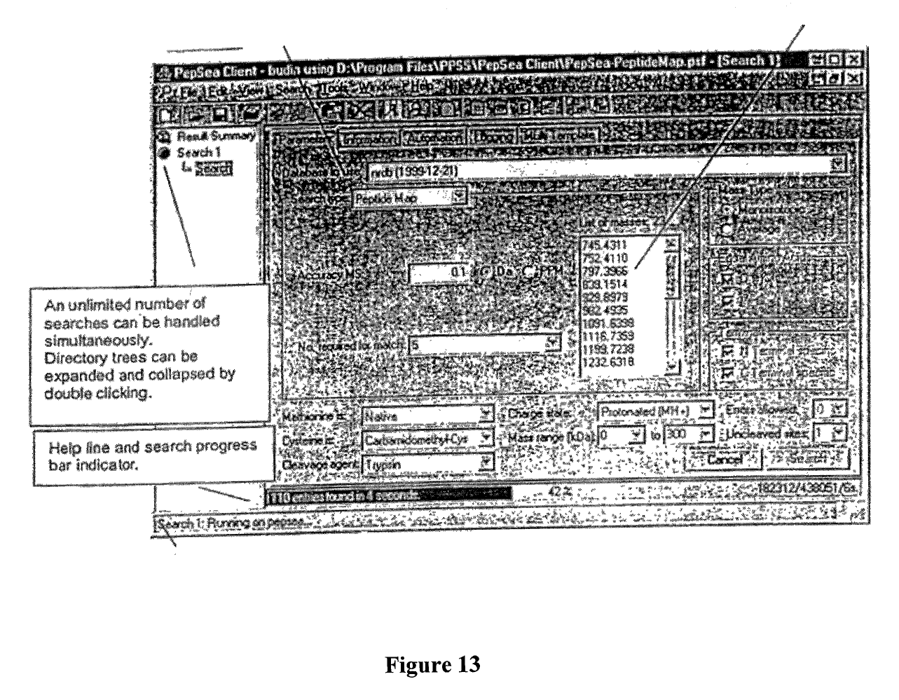

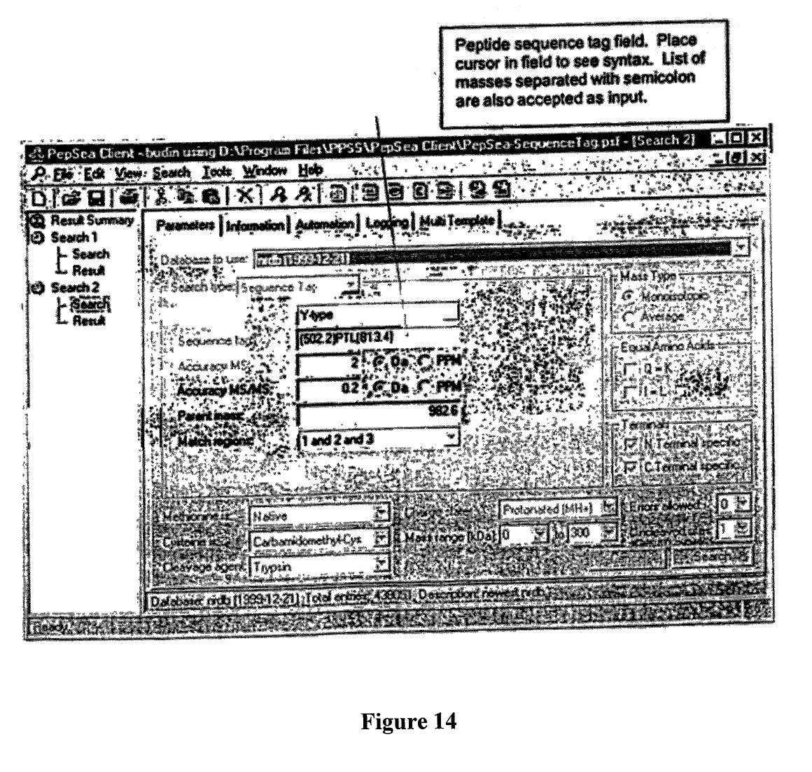

The window in FIGS. 13 and 14 contain the primary information that can be used in the database query, e.g., under the mass spectrometric data. In the illustrated interface, the user can create searches using peptide maps, peptide sequence tags, breakpoint, and sequence alone. Help lines pertaining to any parameter field can be provided, shown here in the lower left-hand corner of the window, e.g., by leaving the cursor over the field of interest.

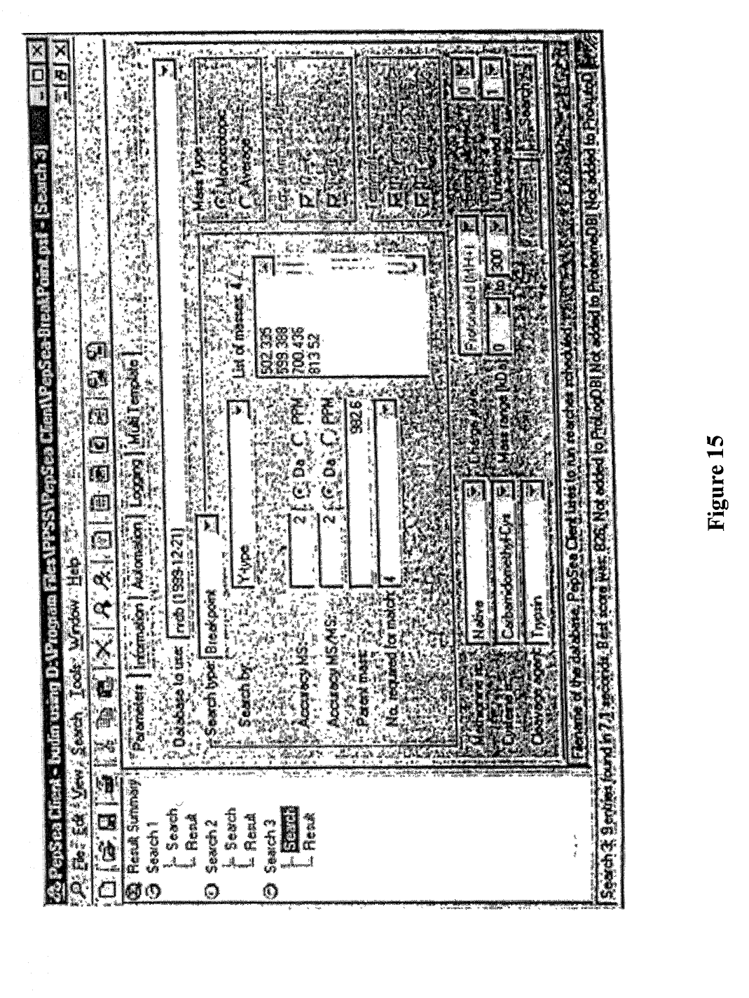

Nucleotide databases can be queried by peptide maps by the ID program version. ‘Breakpoint’ searches require a defined minimum number of fragment ion masses to match theoretically expected fragment masses. See FIG. 15. For example, for a database entry to match, from a list of 10 masses. The system may require that at least 5 of these masses must be possible Y-ions.

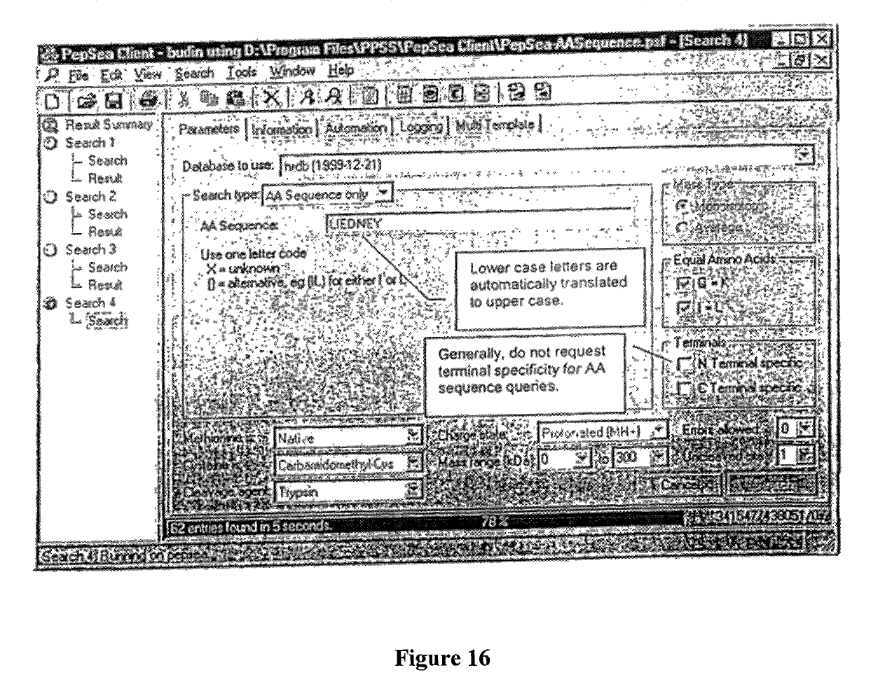

Main parameters are the precursor mass along with a list of fragment masses of which a requested number must theoretically match calculated fragment masses of Y, B, or Y AND B ions. See FIG. 16. The MS error may, for example, be chosen very large (say, 50 Da) to accommodate for modifications and substitutions etc. To regain search specificity, a very small MS/MS error should then be used (for example, 20 ppm).

This search method is useful for searching on completely uninterpreted data. However, the search specificity is not as high as for sequence tag searches.

(vi) Additional Sample Information Dialog Box

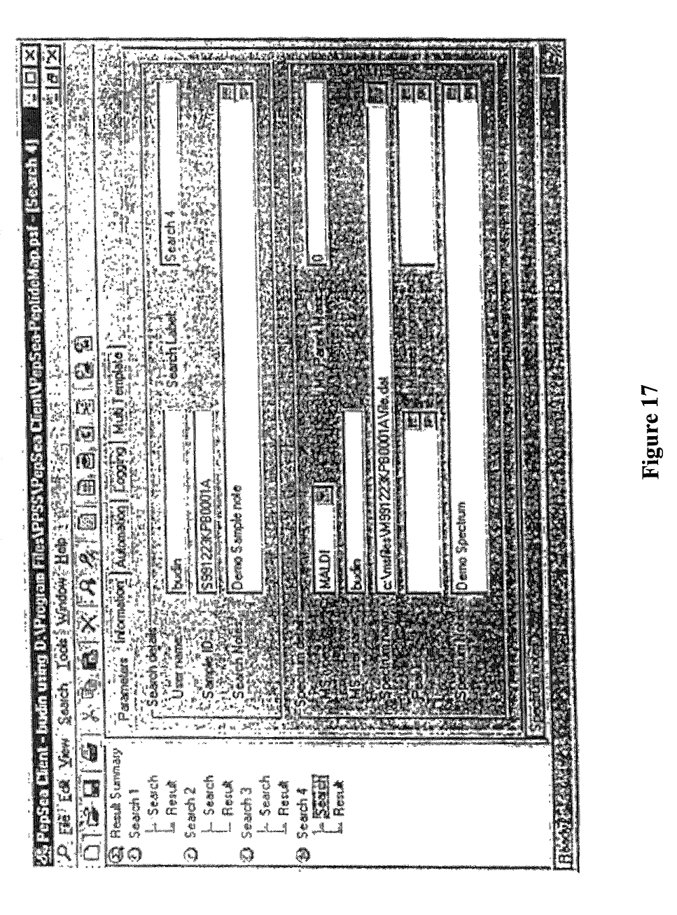

FIG. 17 shows a sample dialog box for entering and viewing information that is secondary to the database searches, i.e. it is unnecessary for the search itself but which may be relevant to the information flow following completion of the search. All of this information is expected to be entered automatically, either by the ID program Client itself or by the ID program Flow Agent parsing information to ID program. In the illustrated case, search information can then be logged in ProteomeDB (if logging is selected) but ONLY if a unique sample record can be assigned. This requires the field ‘Sample ID’ to be filled out correctly. There must also be a spectrum name. Other fields can remain empty and still allow logging.

(vii) Automating ID Program

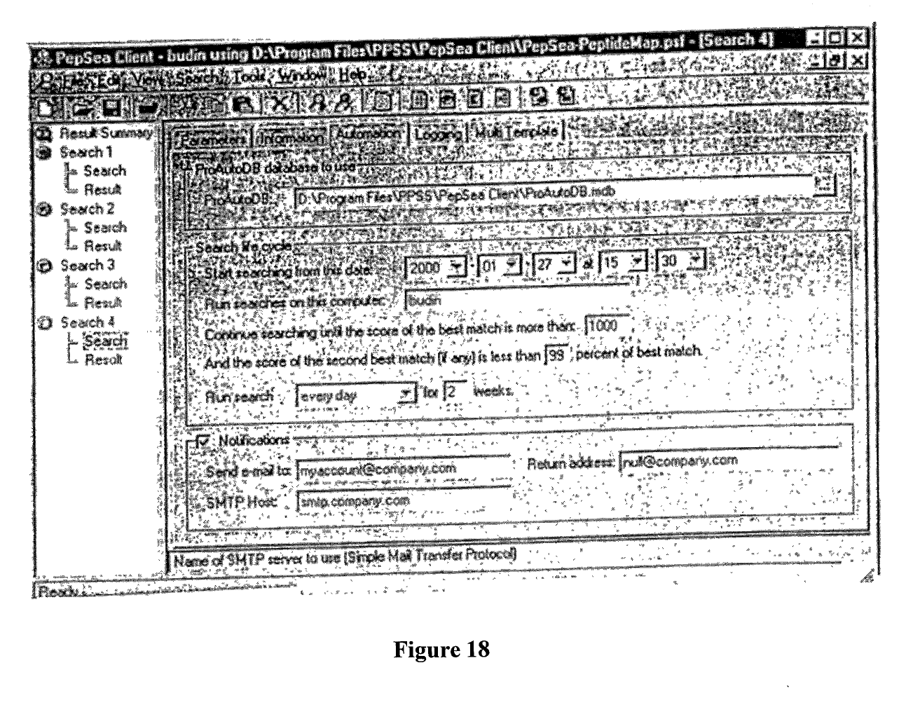

FIG. 18 shows a search parameter window for automating pattern matching. The ‘Search life cycle’ can be set in the Automation tab of the search window. Parameters that may be required by the subject system include:

1. When a new search should be run if the initial search fails; e.g. a certain number of days, next database update, etc.

2. On which computer the search should run.

3. The definition of a failed search. The system may be instructed to continue searching until the score of the best match is more than this value and the score of the second best match (if any) is less than a percentage of the best match. This means that the search will be scheduled if the score of best match was not high enough. A score of 1 means that no searches will be scheduled. A percentage of 99 means that the score of the second match will be ignored.

4. How often the search should be run. For example, the user could specify to run the search once, or each day/week for a number of weeks.

In certain embodiments, the ID program Client can be configured to send an e-mail to a user when a match is found or when the search life cycle has ended and no match has been found. For instance, the ID program Client can use the Simple Mail Transfer Protocol to send an e-mail.

(viii) Logging Searches

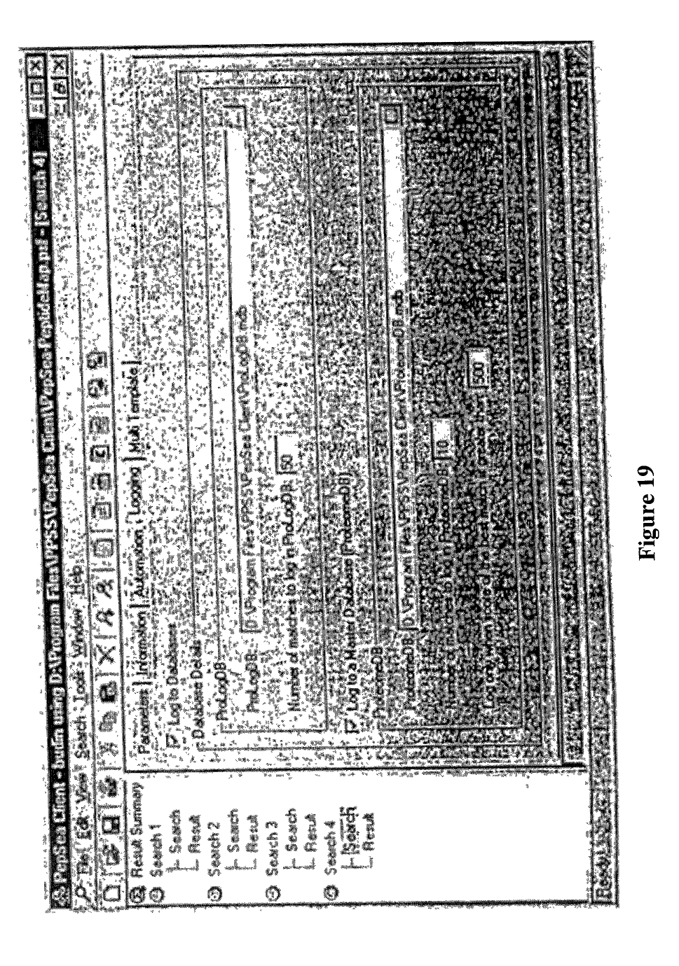

FIG. 19 shows one embodiment in which the options for logging search results can be specified by the user. These log files can be, e.g., local or on a remote file server. If the ProLogDB file does not exist, ID program Client will create such a file.

The ProteomeDB database file can be the file that contains the tables. This means that if a database is split into forms and tables (e.g., by Microsoft Access function) then ID program Client must also keep track of the various parts.

(ix) The Multi Template Interface

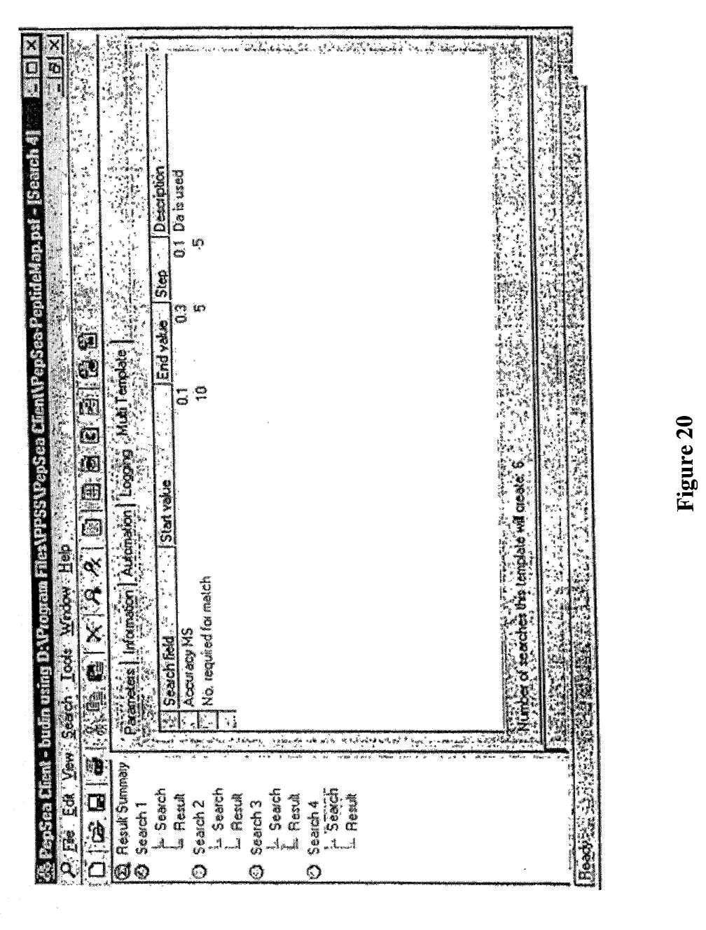

In certain embodiments, it will be desirable to automatically apply other search parameters if the search failed using the standard parameters. See FIG. 20.

In such embodiments, the user may be prompted to set the standard search parameters to the most accurate values. If the standard search fails then the user may define one or more follow-up searches using, e.g., less stringent criteria.

(x) The Search Results Window

Matching entries to a query can be returned in a dynamic table that allows alphabetic sorting in either ascending or descending order following any column contents. Default sorting may follow a score, which follows empirical scoring algorithms based on observations from hundreds of database searches. For example, these may have the form:

Peptide Map:

$$Score = Ks \cdot \frac{{( {\sum }_{n = 1}^{Nm} {( 1 + Ke \cdot {\vert Δ Mn \vert}^{K Δ} )}^{- 1} )}^{2}}{Nu \cdot \sqrt{Mprot}} ,$$

where Ks=1100; Nu=the number of peptide masses entered; Nm=the number of matching masses; Ke=1.0; DMn=the absolute mass error; KD=1.0; and Mprot=protein Mw in KDa

Sequence Tag:

$$Score = \{ \begin{matrix}S2ifS2 \geq 0 \\ 0 if S2 \leq 0\end{matrix} \} ,$$

where S2=S1+Sn+Sc, where Sn and Sc are 0 if N- and C-terminal specificity are false and 100.00 if they are true, respectively.

S1=K max−\sqrt{Ke·ΔMpep}, where Kmax=500; Ke=100; and Dmpep=the absolute mass error.

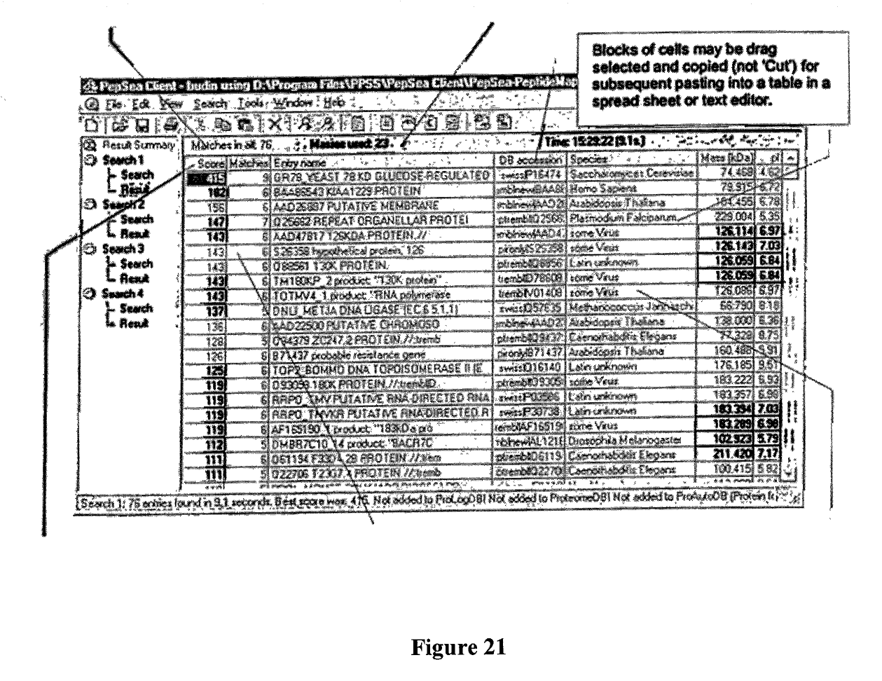

FIG. 21 shows an illustrative Search Result Window. Selecting and then ‘right-clicking’ on an entry in the result window, for example, can bring up a menu with a selection of information windows to further enhance the analysis of that entry.

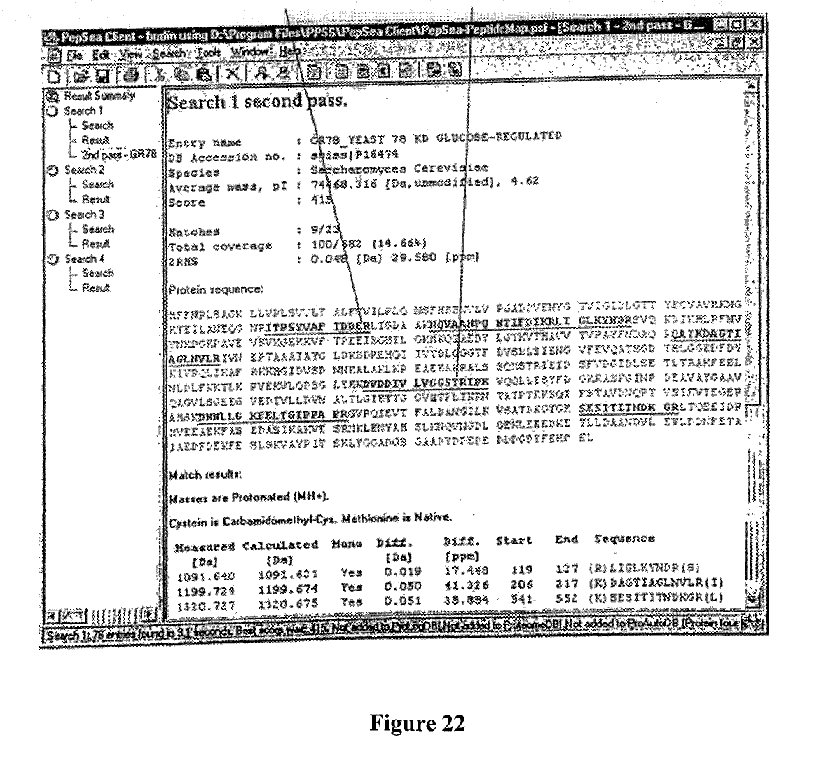

FIG. 22 shows an exemplary 2nd pass check window. To obtain a 2nd pass check of a given matching database entry, the entry can be selected, e.g., by left-clicking in any field its row on the Result window, and then either right-click to choose or go to the side bar to choose the ‘2nd passcheck’ window. The window displays the entire sequence information in the index file with the matched sequence pieces highlighted in different colors. Sequence covered by one matching peptide is a first color, that covered by two peptides is another color, and so forth.

(xi) Database Entry Window

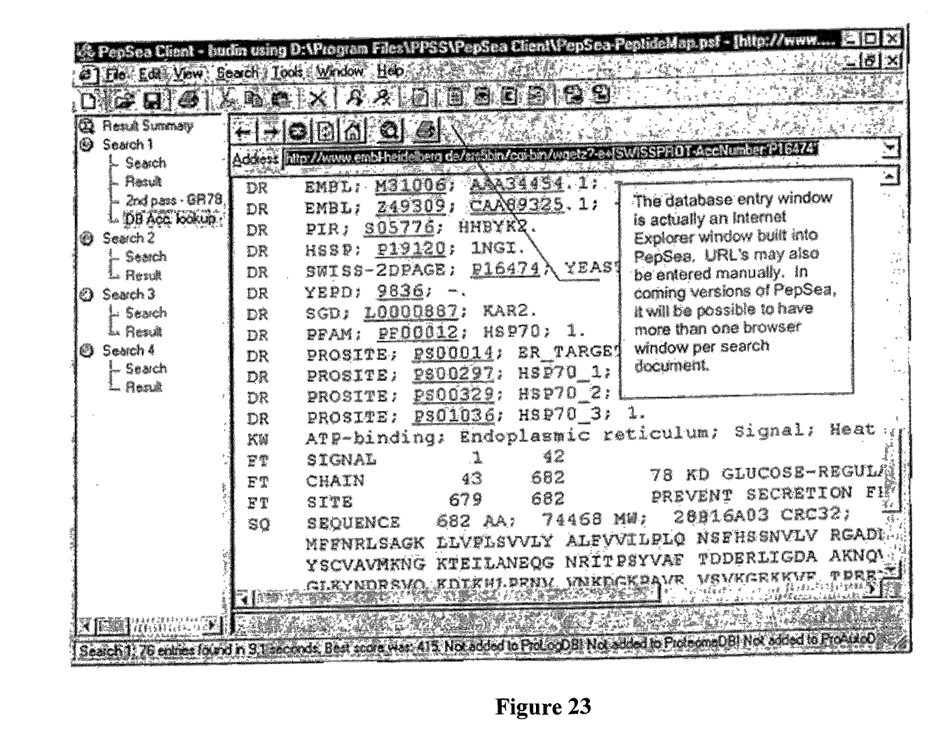

FIG. 23 shows an exemplary database entry window. The illustrated browser window is for fast access to e.g. SwissProt and BLAST searches at NCBI. The addresses of the databases are listed in a settings file and can be changed to utilize Intranet mirrors instead of the presently chosen sites.

(xii) Result Summary List Window

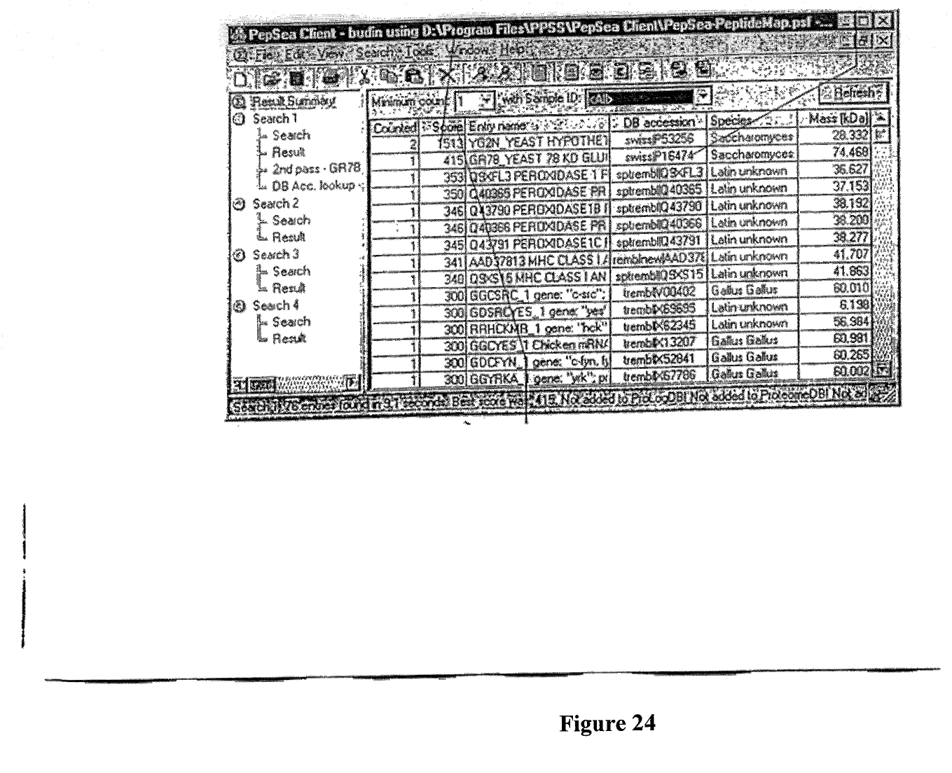

FIG. 24 illustrates what a Result summary window may look like. There is a small check box in the upper right-hand corner of each search result window. When checked, the contents of the individual search result windows are parsed to the summary window. Here the result lists are interleaved to allow the proteins found to multiple times register with counted occurrence and added score values. This is meant to provide a simple and fast means of comparing data from several individually non-specific searches (as arising from short sequence tags and low abundance MALDI maps, for example).

To identify which search(es) a particular entry was found in, the user can select the entry and choose ‘Find result entry’. This will bring the pertinent single result windows to the foreground while highlighting the entry of interest in each list.

(xiii) The Database Browser Window

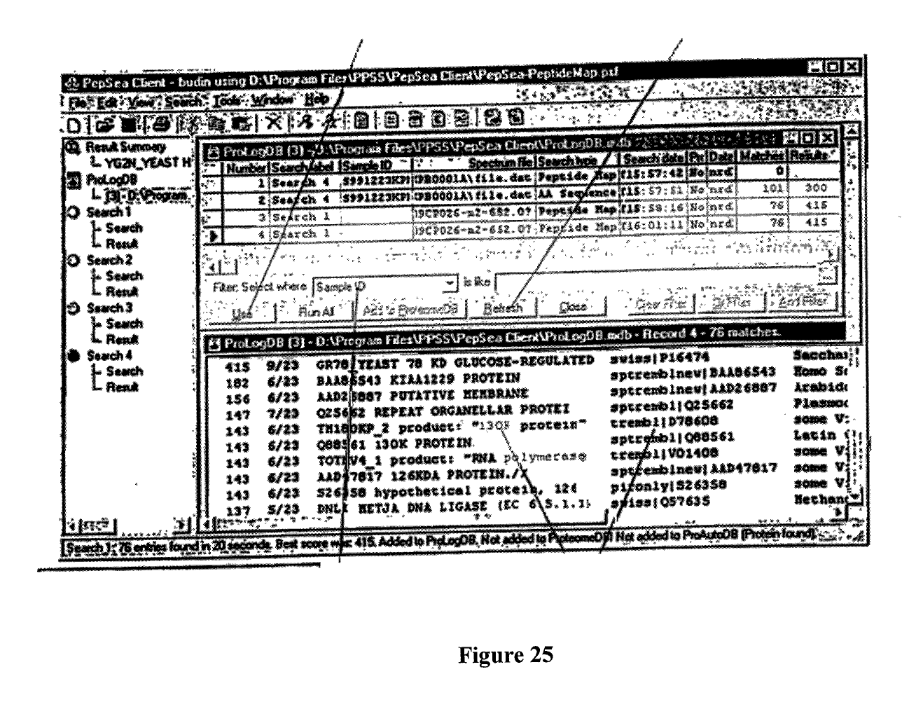

FIG. 25 is an illustration of a database browser window. In this example, the ProLogDB file whose path is specified in the ‘Logging’ tab can be browsed directly from the ID program Client by choosing ‘ProLogDB’ in the ‘View’ menu. The result list can also show in full length in a separate window (not displayed here) whenever a record is selected (highlighted). Alternatively, it is possible to work with the contents of these files via Microsoft Access or the like.

(xiv) Miscellaneous Features

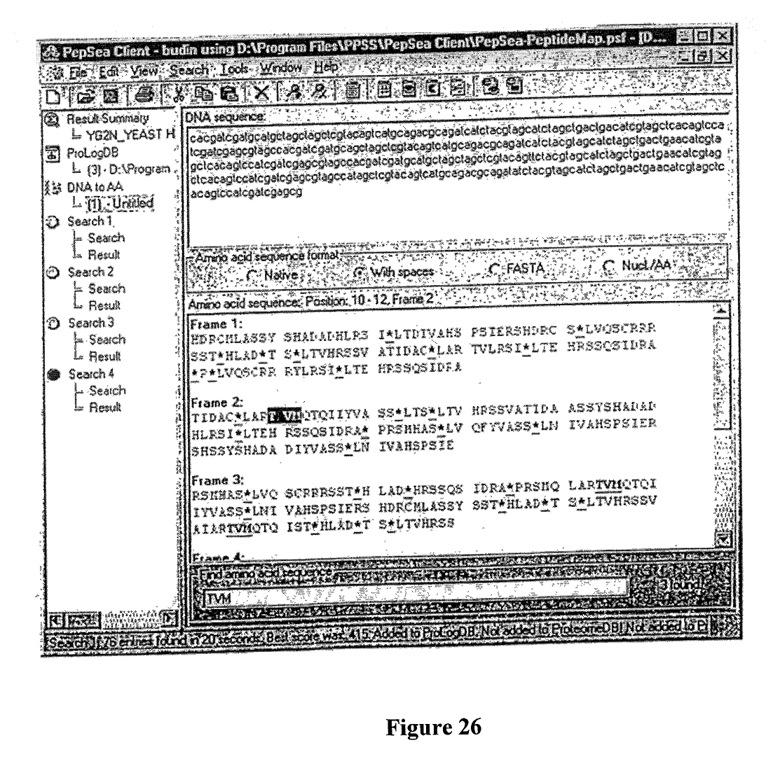

FIG. 26 shows two sample windows for permitting the user to convert a nucleic acid sequence to the corresponding amino acid sequence.

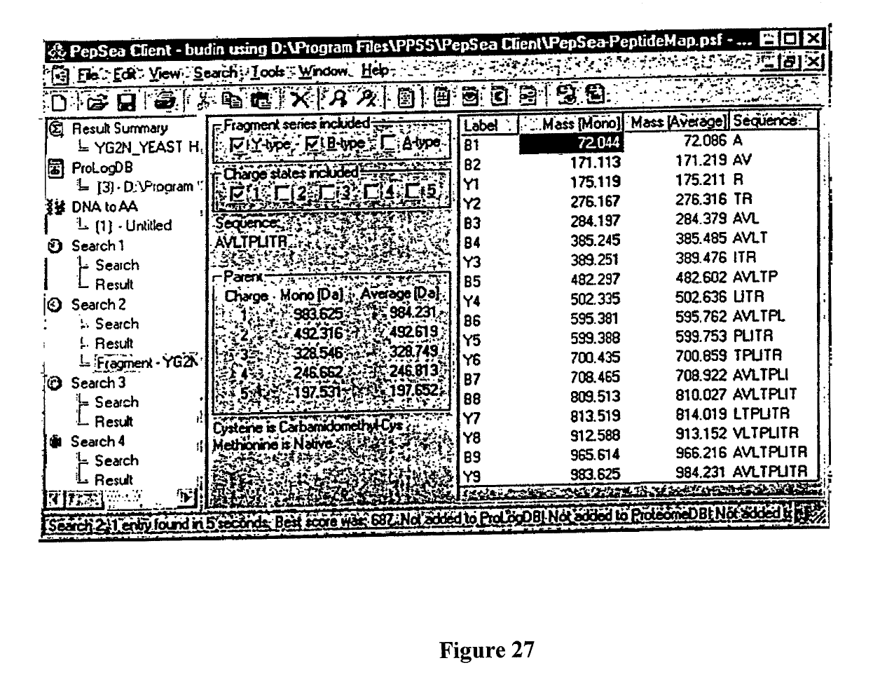

FIG. 27 shows an exemplary search parameter window for calculating theoretical fragment masses from peptide sequences. Selecting an entry from any result window from searches other than on peptide maps will enable the calculation of theoretical fragment mass values. These can be sorted (ascending and descending), for example, by each of the column titles shown in the window below.

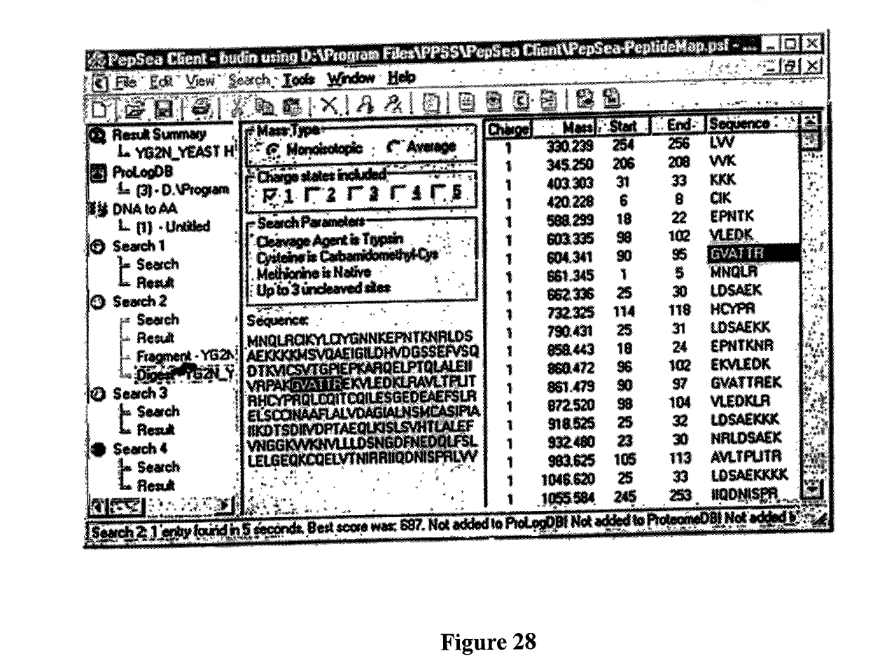

FIG. 28 is a window which can be used as an interface for translating DNA sequence data that is either typed or copied into the window. It is also possible to type in a stretch of amino acid sequence to check for the occurrence of the sequence in each reading frame. This feature can be used for the validation of found ESTs on queries by MS/MS data. However, the window can also be used generally for highlighting amino acids sequence stretches in longer sequences of amino acids copied in (disregarding the use of the translation facility).

(xv) Overview of an ID Program Flow Agent

The ID program Flow Agent can function as conduit for control and information between the mass spectrum acquisition software and ID program by transferring a list of peptide masses from an MS peptide map to ID program client for subsequent database search. The ID program Flow Agent can monitor specified folders for the arrival of new peak lists and then transfers these with or without relevant specific search parameters to ID program. This application is generally useful on computer systems that are directly in control of mass spectrometric data acquisition and handling or wherever the mass spectra are stored, but it also works well over a network.

VII. Exemplification

### Example 1

Proteome projects seek to provide systematic functional analysis of the genes uncovered by genome sequencing initiatives. Mass spectrometric protein identification is a key requirement in these studies but to date, database-searching tools rely on the availability of protein sequences derived from full-length cDNA, expressed sequence tags (ESTs) or predicted open reading frames (ORFs) from genomic sequences. We demonstrate here that proteins can be identified directly in large genomic databases using peptide sequence tags obtained by tandem mass spectrometry. On the background of vast amounts of non-coding DNA sequence, identified peptides localize coding sequences (exons) in a confined region of the genome, which contains the cognate gene. The approach does not require prior information about putative ORFs as predicted by computerized gene finding algorithms. The method scales to the complete human genome and allows identification, mapping, cloning and assistance in gene prediction of any protein for which minimal mass spectrometric information can be obtained. Several novel proteins from A. thaliana and human have been discovered in this way.

A. Materials and Methods

Proteins: Protein samples from Arabidopsis A. thaliana were excised from a 2D PAGE gel of a total membrane-associated protein preparation. (Human protein samples were obtained from ongoing research projects within our group and through collaborations, see Example 2). Spots were excised from gels and digested with trypsin as described previously. See Shevchenko et al. 1996 Anal Chem 68:850-858.

Mass spectrometry: MALDI mass spectra were acquired on a Bruker REFLEX III reflectron time of flight (TOF) mass spectrometer (Bruker-Daltonik, Bremen, Germany). Matrix surfaces were made from α-cyano-4-hydroxycinnamic acid by the fast evaporation method. Vorm et al 1994 Anal. Chem. 66:3281-3287; and Jensen et al. 1996 Rapid Commun Mass Spectrom 10:1371-1378.

About 1-2% (0.3-0.5 μl) of the supernatant of in-gel trypsin digests were injected into an acidified drop previously deposited onto the matrix surface. The monoisotopic masses for all peptide ion signals in the acquired spectra were determined and used for database searching. Peptides were also analyzed by electrospray tandem MS on a prototype quadrupole time-of-flight mass spectrometer (MDS Sciex, Toronto, Canada) equipped with a nanoelectrospray ion source (MDS Protana A/S, Denmark). The mixture of peptides obtained by in-gel proteolysis was purified and concentrated prior to nanoelectrospray MS as described. Shevchenko et al, supra; and Wilm et al. 1996 Anal Chem 68:1-8. For tandem MS experiments, ions were selected using the mass resolving quadrupole. Mass analysis of fragment ions was performed by the TOF analyzer. Peptide sequence tags were constructed from the tandem mass spectra and used for subsequent database searching.

Genome databases and searching: The A. thaliana genome database was obtained from the curators of the Arabidopsis Genome Initiative at The Institute of Genomic Research (TIGR), Rockville, Md.). A custom Perl script was used to convert the downloaded database into a FASTA formatted sequence index file accepted by the PepSea database search software system (MDS Protana A/S, Denmark). The human genome database (HGdB) was constructed in a similar fashion. Finished and unfinished human genome sequences (phases 0-3) were downloaded from the NCBI ftp site. Peptide sequence tags and MALDI peptide mass maps were searched against the respective databases using the program PepSea (MDS Protana A/S, Denmark). Default search criteria specified trypsin as the protease and required measurement accuracy of better than 50 ppm for both intact peptide ion and fragment ion masses. The amino acid part of the peptide sequence tag was translated into the corresponding degenerated oligonucleotide sequence. Potential hits in the forward or reverse direction on the human genome data were checked as to whether they coded for the amino acid sequence of the tag. The mass distance to the N- and C-terminal part of the potential peptide match was then calculated in the reading frame defined by that match. For the A. thaliana genomic database, searches took 2 s to complete on a PC cluster.

Gene prediction: Several web-based gene prediction programs were employed for further characterization of identified coding regions of the A. thaliana and human genome. These included GENSCAN at the Massachusetts Institute of Technology (MIT, Boston, USA), HMMgene at the Center or Biological Sequence Analysis (CBS, The Technical University of Denmark, Lyngby, Denmark) and GRAIL at the Oak Ridge National Laboratory (Oak Ridge, USA).

B. Results and Discussion

In order to assess if protein derived MS data can be utilized for the identification of proteins in genomes of higher organisms, we analyzed a large number of human and Arabidopsis thaliana (A. thaliana) proteins by nanoelectrospray tandem mass spectrometry and subsequent interrogation of genome sequence databases using the mass spectrometric data.

(i) Identification of Proteins in the A. thaliana Genome

The sequenced region of the 125-megabase A. thaliana genome covers 115.4 megabases. A combination of algorithms has been used to predict 25,498 putative genes consisting of 132,982 exons with a total length of 33,249,250 bases corresponding to 29% coding bases (Initiative 2000 Nature 408:796-815). From a single two dimensional gel (supplementary material) of a total membrane associated protein preparation of A. thaliana, 60 spots were analyzed by a two step mass spectrometric procedure (Shevchenko et al. 1996 PNAS 93:14440-14445.) consisting of mass fingerprinting followed by tandem mass spectrometric peptide sequencing using nanoelectrospray (Wilm et al., supra; and Wilm et al. 1996 Nature 379:466-9).

At the time of the experiment, approximately 75% of the A. thaliana genome was publicly available. Fifty-one of the 60 proteins analyzed were identified in amino acid sequence databases (48 by mass fingerprinting, three by tandem mass spectrometry; see supplementary information for details) and nine were novel proteins. Attempts to identify proteins in the A. thaliana genome by peptide mass fingerprinting alone failed because many genomic regions, when translated in the three forward or reverse reading frames, give rise to a significant number of randomly matching peptide masses. Instead, tandem mass spectrometric data of a test set of 20 spots consisting of eleven identified and all nine novel proteins were assembled into peptide sequence tags (Mann et al. 1994 Anal Chem 66:4390-4399) and used to search the genomic database of A. thaliana. A peptide sequence tag consists of a few amino acids that can easily be assigned (manually or by software) in a tandem mass spectrum. These amino acids are ‘locked’ into position within the peptide by the ‘start’ and ‘end’ masses of a fragment ion series. Together with the mass of the intact peptide, a search template is created. The use of accurate peptide and fragment ion mass information in addition to amino acid sequence information increases the search specificity of a peptide sequence tag by more than a million fold over the short amino acid tag sequence alone. Searches using the peptide sequence tag algorithm (Mann et al. 1994 Anal Chem 66:4390-4399) were performed directly on the genomic data without prior translation of the genome sequences into amino acid sequences (the search is performed once in the forward and reverse directions of the nucleotide sequence) and without regard for predicted coding regions or reading frames. Peptide sequence tags containing as few as two or three amino acids almost always identified a single location in the A. thaliana genome. The specificity of the peptide sequence tags is aided by the high mass accuracy (between 5 and 50 ppm depending on signal strength) provided by quadrupole time of flight instruments even on femtomole amounts of gel separated proteins. Morris et al. 1996 Rapid Commun Mass Spectrom 10:889-896; and Shevchenko et al. 1997 Rapid Commun Mass Spectrom 11:1015-1024. Because the full retrieved peptide sequence is verified against the fragmentation spectrum, the specificity of a peptide sequence tag search approximates that of the corresponding full peptide sequence. In all cases, proteins could uniquely be identified by a combination of two peptides because they resulted in hits within a confined region of the genome. Four peptide sequence tags were observed to cluster in a 2 kb region of the A. thaliana genome. Whenever a peptide sequence tag unambiguously identifies the corresponding DNA sequence in the genome, this sequence must be part of an exon. The peptide therefore locates the exon and establishes the correct reading frame. In-frame stop codons upstream and downstream of the identified peptide also limit the extent of the exon within which the splice signals (exon intron boundaries) must be found. This information is useful for the reconstruction of the gene from the nucleotide sequence (see below).

Of the eleven known proteins in the test set, seven were unambiguously identified in the A. thaliana genome. Four proteins did not result in a hit in the 75% genomic sequence available, consistent with the result of searching their known sequences in that database which revealed that they were not yet present. Of the nine novel proteins, we identified five in the A. thaliana genome whereas the remaining four, despite high quality mass spectrometric data, did not result in a match in the database. We therefore concluded that these proteins were not yet present in the genome database (Table 1). During the preparation of this manuscript, the A. thaliana genome was published. Initiative, supra. Searching the previously unidentified data in the complete A. thaliana genome, yielded the identification of all the novel proteins, indicating a 100% success rate of the method presented here.

Unambiguous identification of peptides in the genome directly provides the information necessary for further analysis of the corresponding gene. The identified exon sequences define the direction of the nucleotide sequence. The identified exon can be used directly for homology searching, and as probes for cloning the genes. Furthermore, the above identifications map the respective genes to their locations in the genome.

Once part and direction of the coding sequence of a protein had been found in the genome by peptide sequence tags, the mass fingerprinting data obtained in the first step of the mass spectrometric analysis was used to obtain further information about the gene structure. Since only peptide masses are available in peptide mass mapping, the identified genomic region (approximately 10 kb for A. thaliana) was translated in three reading frames. The exon sequence coverage can be refined and additional exons can sometimes be discovered by peptide mass mapping.

Peptide sequences can also be used to join adjacent exons. For example, the underlined part of the peptide sequence TFDESKETINKEIEEK (SEQ ID No: 1), derived from MS sequencing of the protein S8, is located in exon 1 and the remainder in exon 2 (Table 1) of the gene. For proteins from A. thaliana, these ‘peptide exon bridges’ were frequently found (on average one per protein) but not as frequent as to always allow the full reconstruction of the gene.

While gene prediction in genomic DNA of higher organisms is difficult, a combination of mass spectrometric data and computational exon prediction can be very effective at defining gene structure. The genomic region identified through MS data for spot S8 was analyzed with GENSCAN, HMMgene and GRAIL and compared to the known sequence of this protein. Both predictions by GENSCAN and HMMgene missed one exon and predicted a surplus exon whereas GRAIL predicted several splice sites incorrectly. However, when the peptides identified by MS were used as constraints for coding sequence prediction (using HMMgene), the surplus exon was no longer predicted and the previously missed exon was included (though still with a splice site error). Mass spectrometric data, together with the genome sequence, rectified all incorrect splice sites, led to inclusion of the complete exon that was missed previously and showed that the surplus exon was not present.

While useful in many cases, the extent to which a predicted gene model can be verified or refined by the MS data obviously depends on the number of exons actually identified by peptide sequence tags. We now perform database searches in real time during the mass spectrometric experiment, combine it with gene prediction to increase the number of identified exons by sequencing of the appropriate peptides and to directly sequence potential exon spanning peptides.

(ii) Identification of Proteins in the Human Genome

The size of the human genome is approximately 25 times that of A. thaliana and it is estimated that only 3% of the nucleotide sequence is coding for proteins. To learn about the feasibility of identifying coding sequences in the human genome on the background of the vast amounts of non-coding sequence, we searched data from more than 200 peptides which we have sequenced by mass spectrometry in various projects against an estimated 80% of the human genome which was publicly available at the time of writing. The results of these experiments are summarized in table 2. Peptide sequence tags comprising four amino acid residues retrieve only a single entry if the peptide is indeed in the human genome and none if it is not. With a three amino acid tag, the search retrieves on average two sequences, only one of which fits the spectrum when comparing all calculated fragment ion masses for the retrieved peptides with the experimental spectrum. With a two amino acid tag, on average seven peptide sequences are retrieved. Evaluation of the sequences yields a unique result in almost all cases except when the peptide is too short to be unique in the database (<10 amino acids). Incidentally, we found that tryptic peptides encountered in MS sequencing are typically longer than 10 amino acids and thus were almost always unique in the human genome. As in the case of searches in the AT genome, however, data from any two peptides was always sufficient for an unambiguous localization of the protein in the genome. This is because it is extremely unlikely that any of the few retrieved sequences, even from short peptides, happen to ‘co-localize’ in the same gene on a chromosome by chance. Still, we recommend the use of at least two peptides per human protein because we find that there is about a 25% chance for a peptide to be spanning an intron exon boundary. Such peptide exon bridges can be detected if another peptide has previously identified the general location of the gene. We are currently working on software that will allow the identification of peptide exon bridges in the complete genome.

The evaluation, or matching, of the retrieved sequences against the mass spectrum, while unambiguous, was done manually in this project. However, we have investigated the use of an objective criterion to match the spectra per computer. We have found that the ratio of fragments that additionally match the tandem mass spectrum is at least twice as high for the correct sequence than it is for sequences that only matched the formal sequence tag criteria. We also found that fragment ions that continue a tag series were particularly powerful for discrimination because a fragment which continues an ion series effectively converts a tag with n amino acids into one with n+1, leading to reduced matches as shown in Table 2.

Altogether, we searched experimental data against the human genome database from 49 human proteins from ongoing projects where we had tandem mass spectral data on at least two peptides. Of the 49 proteins, 41 (84%) were unambiguously found in the genome by the methods and criteria explained above. Eight proteins (or 16%) of the proteins were unambiguously found not to be contained in the version of the public database that we searched. That version is estimated to contain 80% of the human genome in various states of sequence quality refinement. However, we found all of the eight proteins in non-redundant protein or EST databases by using the mass spectrometric data. We then performed BLAST analysis of these sequences against the human genome which confirmed that they were not yet present in the genome. Hence, like in the case of searching the A. thaliana genome, the success rate of searching the human genome with MS data was also 100% and shows that the protein identification method described here scales to very large genomes with a low proportion of coding sequence.

(iii) Identification of a Novel Human Protein

Data obtained in our group is now routinely used to identify human proteins in the genome database using the methodology presented in this paper. As an example, we were able to identify a novel human protein starting from a weak silver stained spot from a two dimensional gel. The spot turned out to consist of mixtures of proteins, three known proteins were found in a protein sequence database (data not shown). One additional protein was identified in the human genome database as follows. Four peptide sequence tags were obtained by nanoelectrospray tandem mass spectrometry (Table 3) and queried against the human genome database. Their matches formed a cluster located in two exons and the identification was confirmed by corresponding peptide signals in the peptide mass fingerprint. The gene model proposed by both GENSCAN and HMMgene of the genomic region containing the two exons was compatible with all MS data. Sequence analysis of the identified protein revealed the presence of a signal sequence at the N-terminus of the proteins as well as a Jacalin domain (PFM), which is also found in animal prostatic spermine binding proteins.

The methods presented here can be used in small and large-scale proteomics projects for all organisms that have sequenced genomes as well as their close relatives. Given the availability of minimal mass spectrometric peptide fragmentation data, it is possible to identify any protein from those organisms whether or not additional sequence information in the form of ESTs is available. The approach does not rely on a completely assembled genome sequence, only on full coverage of the genome, which, to date, can be achieved relatively quickly even for large genomes. Furthermore, together with ongoing bioinformatics and comparative genomics efforts, existing EST projects and planned full-length cDNA projects, mass spectrometry combined with genome searches will play a valuable tool for discovering and characterizing the proteins coded by the human and other genomes and provide direct access to cloning of those molecules.

TABLE 1

Peptide sequence tag identification of proteins from A. Thaliana
*Peptide sequences are the result of searches by peptide sequence tags in the
A. thaliana genome. For all hits in the genome data, further peptides were
identified using the MALDI MS peptide mass map (data not shown).
Proteins that did not result in a hit in the 75% genomic sequence or the
non-redundant protein database available at the time of the experiment.

| | | | |
|---|---|---|---|
| | Apparent | NRDB accession | |
| Protein | Mw [kDa]/pI | No. | Sequences identified in genome |

| | | | | | |
|---|---|---|---|---|---|
| 1 | (S11) | 30/5.5 | tnew AC007019 | FQAAVDILR; | (SEQ ID No: 2) |
| | | | IKHDIDTETQDIPDAR; | (SEQ ID No: 3) | |
| | | | ITLDPEDPAAVK; | (SEQ ID No: 4) | |
| | | | VFFDIK; | (SEQ ID No: 5) | |
| | | | AQLDELKSDAVEAMESQK; | (SEQ ID No: 6) | |
| | | | SDKKGMDLLVAEFEK; | (SEQ ID No: 7) | |
| | | | KEDLPKYEENLELSMAK | (SEQ ID No: 8) | |
| 2 | (S8) | 44/4.8 | spt Q96262 | YLEELVK; | (SEQ ID No: 9) |
| | | | VSVFLPEEVK; | (SEQ ID No: 10) | |
| | | | VVETYEATSAEVK; | (SEQ ID No: 11) | |
| | | | EIPVEEVKAEEPAK | (SEQ ID No: 12) | |
| 3 | (S10) | 55/6.6 | spt 39206 | AIAFDEIDKAPEEK; | (SEQ ID No: 13) |
| | | | FPGDDIPIIR; | (SEQ ID No: 14) | |
| | | | VGEEVEILGLR; | (SEQ ID No: 15) | |
| | | | GSALSALQGTNDEIGR; | (SEQ ID No: 16) | |
| | | | LMDAVDEYIPDPVR | (SEQ ID No: 17) | |
| 4 | (S5) | 60/4.3 | spt 004151 | TLVFQFSVK; | (SEQ ID No: 18) |
| | | | FYAISAEFPEFSNK | (SEQ ID No: 19) | |
| | | | FYAISAEFPEFSNKDK | (SEQ ID No: 20) | |
| 5 | (S12) | 65/5.1 | CAB37531 | AVVTVPAYFNDAQR; | (SEQ ID No: 21) |
| | | | EVDEVLLVGGMTR; | (SEQ ID No: 22) | |
| | | | GVNPDEAVAMGAAIQGGILR | (SEQ ID No: 23) | |
| 6 | (S6a) | 74/5.0 | spt Q39042 | LVPYQIVNK; | (SEQ ID No: 24) |
| | | | DAGVIAGLNVAR; | (SEQ ID No: 25) | |
| | | | FDLTGVPPAPR; | (SEQ ID No: 26) | |
| | | | FEELNNDLFR; | (SEQ ID No: 27) | |
| | | | IMEYFIK | (SEQ ID No: 28) | |
| 7 | (S44) | 100/6.4 | sptnew AAD25640 | ILLESAIR; | (SEQ ID No: 29) |
| | | | TSLAPGSGVVTK; | (SEQ ID No: 30) | |
| | | | FYSLPALNDPR; | (SEQ ID No: 31) | |
| | | | VVNFSFDGQPAELK; | (SEQ ID No: 32) | |
| | | | SENAVQANMELEFQR | (SEQ ID No: 33 | |
| 8 | (S183) | 12/10.0 | swiss P34893 (a) | VIAVGPGSR; | (SEQ ID No: 34) |
| | | | EGDTVLLPEYGGTQVK; | (SEQ ID No: 35) | |
| | | | DEDVLGTLHED; | (SEQ ID No: 36) | |
| | | | TESGILLPEK; | (SEQ ID No: 37) | |
| | | | VIQPAKTESGILLPEK; | (SEQ ID No: 38) | |
| | | | LIPVSVKEGDTVLLPEYGGTQVK | (SEQ ID No: 39) | |
| 9 | (S2) | 55/5.7 | swiss P29685 | TIAMDGTEGLVR; | (SEQ ID No: 40) |
| | | | VVDLLAPYQR; | (SEQ ID No: 41) | |
| | | | IGLFGGAGVGK; | (SEQ ID No: 42) | |
| | | | VGLTGLTVAEYFR | (SEQ ID No: 43) | |
| 10 | (S4e) | 62/10.2 | spt O23656 (a) | FGLYYVDFK; | (SEQ ID No: 44) |
| | | | EYADYVFTEYGGK; | (SEQ ID No: 45) | |
| | | | LSIAWSR; | (SEQ ID No: 46) | |
| | | | IGIAHSPAWFEPHDLK | (SEQ ID No: 47) | |
| 11 | (S4b) | 64/8.5 | spt Q42585 (a) | EYADFVFQEYGGK; | (SEQ ID No: 48) |
| | | | DFLSQGVRPSALK; | (SEQ ID No: 49) | |
| | | | FGLYYVDFK; | (SEQ ID No: 50) | |
| | | | NLNTDAFR | (SEQ ID No: 51) | |
| 12 | (S172) | 25/4.3 | spt Q9LW15 (b) | LDDIDFPEGPFGTK; | (SEQ ID No: 52) |
| | | | SYYDKR | (SEQ ID No: 53) | |
| 13 | (S152) | 32/8.5 | spt BAB10927 (b) | TLMNVFDK; | (SEQ ID No: 54) |
| | | | TLMNVFDKTPNVDK; | (SEQ ID No: 55) | |
| | | | VFFSSSAVEYSNLAQAHATENAK | (SEQ ID No: 56 | |
| 14 | (S18) | 36/4.5 | spt Q9LTJ7 (b) | TVVDKSDDAPAETVLK; | (SEQ ID No: 57) |
| | | | QYDGSDPQKPLLMAIK | (SEQ ID No: 58) | |
| 15 | (S106) | 80/4.8 | spt 19S7E7 (b) | FWDNFGK; | (SEQ ID No: 59) |
| | | | FGWSANMER; | (SEQ ID No: 60) | |
| | | | YLSVTNPELSK; | (SEQ ID No: 61) | |
| | | | IYEMMDVALSGK; | (SEQ ID No: 62) | |
| | | | EVTTAEYNEFYR; | (SEQ ID No: 63) | |
| | | | AQSTGDTISLDYMK | (SEQ ID No: 64) | |
| 16 | (S104) | 90/5.2 | spt BAB09837 (b) | IFGEDFLNDK; | (SEQ ID No: 65) |
| | | | SIDSLVITK; | (SEQ ID No: 66) | |
| | | | YFFDGEIQSDKIK; | (SEQ ID No: 67) | |
| | | | VLTEFQEAAK; | (SEQ ID No: 68) | |
| | | | YFFDGEIQSDK; | (SEQ ID No: 69) | |
| | | | IDATEENELAQEYR; | (SEQ ID No: 70) | |
| | | | AEDDVNFYQTVNPDVAK | (SEQ ID No: 71) | |
| 17 | (S93) | 21/7.9 | spt Q9M7T0 (a, b) | LAEGTDITSAAPGVSLQK; | (SEQ ID No: 72) |
| | | | AVNVEEAPSDFK; | (SEQ ID No: 73) | |
| | | | WSAYVEDGKVK; | (SEQ ID No: 74) | |
| | | | SKLAEGTDITSAAPGVSLQK | (SEQ ID No: 75) | |
| 18 | (S36) | 34/8.7 | spt AAG12816 (a, b) | NYINLAQIHASENSK; | (SEQ ID No: 76) |
| | | | SFEQIEVER; | (SEQ ID No: 77) | |
| | | | INLAQIHASENSK; | (SEQ ID No: 78) | |
| | | | AIYTVGNWIR | (SEQ ID No: 79) | |
| 19 | (S7) | 56/7.3 | spt Q9SGA7 (a, b) | IPTAELFAR; | (SEQ ID No: 80) |
| | | | RIPTAELFAR; | (SEQ ID No: 81) | |
| | | | ALEEEIEDIGGHLNAYTSR | (SEQ ID No: 82) | |
| | | | TILGPAQNVK; | (SEQ ID No: 83) | |
| | | | LSSDPTTSQLVANEPASFTGSEVR | (SEQ ID No: 84) | |
| 20 | (S1) | 100/5.9 | spt Q9SSG3 (a, b) | SASITGGYFYR | (SEQ ID No: 85) |

TABLE 2
Statistics of human genome searches with Peptide Sequence Tags

| No of | No of | Hits in | | Unique identification after |
|---|---|---|---|---|
| AA in tag | samples | genome | Range | data verification |
| 2 | 15 | 7.3 | 1-22 | yes for all |
| 3 | 45 | 2.1 | 1-10 | yes for all |
| 4 | 21 | 1.1 | 1-2 | yes for all |
| 5 | 2 | 1.0 | 1-1 | yes for all |

Statistics on a number of searches with peptide sequence tags with various lengths. Peptides were at least 10 amino acids long.

TABLE 3

Mass spectrometric identification of peptides of a
new human protein in the human genome database
Peptide sequence tags were constructed from
nanoelectrospray tandem mass spectra and searched
against the human genome database and the
retrieved peptide sequences are listed.

| | |
|---|---|
| | Sequence identified |
| Peptide sequence tag | in human genome |
| (642.49)VS(99.05) | VSVGLLLVK |
| | (SEQ ID No: 86) |
| (634.38)FAV(246.15) | VFVAFQAFLR |
| | (SEQ ID No: 87) |
| (1347.68)TTSF(163.08) | YFSTTEDYDHEITGLR |
| (SEQ ID No: 88) | (SEQ ID No: 89) |
| (807.45)QLT(1093.57) | LGALGGNTQEVTLQPGEYITK |
| | (SEQ ID No: 90) |

### Example 2

Human proteins obtained from 1D or 2D PAGE gels were digested in gel and the resulting peptide mixtures analyzed by MALDI peptide mass mapping (Bruker Reflex III). Peptides were also sequenced by nanoelectrospray on a quadrupole TOF MS (QSTAR, PE Sciex). Peptide sequence tags and peptide mass maps were searched at 50 ppm mass accuracy against publicly available sequences of the human genome (ca. 80% coverage, NCBI) using the program ID program (MDS Protana). Using a PC cluster consisting of 12 members, searches in the human genome required 75 s CPU time. Further analysis of identified coding regions was performed using the gene prediction programs Grail, HMMgene and Genscan.

Peptide sequences correspond to coding regions within a gene. Whenever a peptide sequence tag derived from a MS/MS spectrum unambiguously identifies the corresponding DNA sequence in the genome, this sequence must be part of an exon. The peptide therefore locates the exon as well as the correct reading frame. In-frame stop codons upstream and downstream of the identified peptide also limit the extent of the exon within which the splice signals (exon intron boundaries) must be found. Mass spectral data can be used to screen the vicinity of mapped regions for further exons. In many cases, peptides span two exons which enables the localization of the exact splice site for the two exons involved.

Typically, several peptides are partially sequenced during the course of a protein identification experiment using nanoES tandem MS. Subsequent database searches identify peptides which cluster in a confined (2-15 kb) region of the genome which encompasses the underlying gene. The identified peptides define reading frames which in turn hold information about the intron/exon structure of the gene. Generally, two peptides are sufficient to identify and map the respective gene to its chromosomal location. Any of the identified exons can be used as probes for cloning or for homology searching for tentative function assignment. The defined genome area can be used to direct sequencing of further peptides in the same experiment.

Most strategies for large scale protein identification follow a two tier analytical approach in which first a MALDI peptide mass fingerprint is created and samples that are not identified in this round of analysis are subjected to partial sequencing by tandem MS. It should be stressed that, owing to the complex structure of genes in higher organisms, fingerprint data alone does not hold sufficient discriminating power to identify proteins directly in a genome. However, once part of the coding sequence of a protein has been found in the genome by peptide sequence tags, the 2-15 kb genomic sequence can be searched with the fingerprinting data by translating the nucleotide sequence in the three respective reading frames. Thereby, exon sequence coverage can be extended and additional exons can sometimes be found.

Computational gene prediction in genomic DNA of higher organisms is very difficult but a combination of MS data and exon prediction can be very effective at defining gene structure. The genomic region identified in the previous figure was analyzed with GENSCAN, and GRAIL and compared to the known sequence of this protein. GENSCAN missed one exon and predicted a surplus one whereas GRAIL predicted two splice sites incorrectly. MS data, in conjunction with the genome sequence, rectified the incorrect splice sites, led to inclusion of the exon that was missed and showed that the surplus exon was not present. The extent to which a predicted gene model can be verified or refined by the MS data obviously depends on the number of exons actually identified by peptide sequence tags.

The size of the human genome is approximately 25 times that of A. thaliana but the coding sequence is expected to be only 2-3 times larger. Tryptic peptides of the size typically encountered in MS sequencing (>10 aa) are almost always unique in the human genome. The information content of peptide sequence tags approximates that of the complete peptide sequence. In addition, the sequences retrieved by the search are checked against the tandem MS data which eliminates false positives. Therefore, searches using even short tags almost always result in unique identifications. Interestingly, the search specificity in the human genome is virtually identical to that of the dbEST but with the added advantage of high sequence accuracy, low redundancy and unbiased coverage.

We claim:

1. A method for identifying a coding sequence in a genomic database, comprising:
(i) generating, for an input polypeptide sequence, a set of sequence tags corresponding to possible coding sequences for the input polypeptide sequence; and
(ii) identifying, by an approximate string matching method using said sequence tags, genomic sequences from a genomic database which are similar to one or more of the sequence tags.

2. The method of any of claims 1, wherein the genomic database is an unannotated genomic database.

3. The method of any of claims 1, further comprising determining an open reading frame for the input polypeptide sequence in the genomic database, and, optionally, determining intron/exon boundaries in the open reading frame.

4. The method of any of claims 1, 2 or 3, further comprising providing annotation for the genomic database.

5. The method of claim 1, wherein the input polypeptide sequence is provided from a system for protein sequencing by mass spectrometry.

6. The method of claim 5, wherein the input polypeptide sequence is provided by a computer which has a data link from a mass spectrometer system for transmitting the input polypeptide sequence.

7. The method of claim 1, wherein the approximate string matching method is selected from: a Shift-And method, a Karp-Rabin fingerprint method, or a Commentz-Walter method.

8. The method of claim 1, wherein the approximate string matching method is a GREP method.

9. The method of claim 8, wherein the approximate string matching method is an AGREP method.

10. The method of any of claims 1, 7, 8 or 9, wherein the approximate string matching method tolerates a maximal number of errors.

11. The method of claim 10, wherein the method tolerates gaps for intronic sequence of a size equal to at least the average length of intronic sequences in the genomic database.

12. The method of claim 10, wherein the error ratio, α, is less than 3.0.

13. The method of claim 10, wherein the error ratio, α, is less than 1.0.

14. The method of claim 1, wherein multiple sequence tags are combined into a single array which is used as the input for the approximate string matching method.

15. A method for identifying a coding sequence in an unannotated genomic database, comprising:
(i) receiving an input polypeptide sequence; and
(ii) identifying, by an approximate string matching method using said input polypeptide sequence, coding sequences from a genomic database which has been dynamically translated in at least 3 reading frames.

16. A computer system for identifying coding sequences in genomic databases, comprising:
(i) a sub-system for calculating and/or storing potential coding sequences for a polypeptide;
(ii) one or more databases of genomic sequence; and
(iii) an ID program for performing approximate string matching between nucleic acid sequences in a manner which accounts for differences between the two sequences due to an intronic sequence;
wherein, the system generates a set of sequence tags corresponding to possible coding sequences for an input polypeptide sequence, and identifies, from the database, any genomic sequences which are similar to one or more of the sequence tags, and indicates exon/intron boundaries, if any, in the genomic sequence(s).

17. The computer system of claim 16, further including a sample/identification proteomics database for logging and correlating information.

18. The computer system of claim 17, wherein said information is one or more of: sample identity, gel photos, mass spectra (and features therein), and search results.

19. The computer system of claim 16, further including a sub-system to automate the transfer and utilization of mass spectrometric data of a target polypeptide.

20. A mass spectrometry system including the computer system of any of claims 16-19, and a mass spectrometer for sequencing polypeptides.

21. The mass spectrometry system of claim 20, wherein the spectrometer includes an ion source selected from: electrospray or MALDI.

22. A method of conducting a proteomics business, comprising:
(i) by the method of claim 1 or 15, determining the identity of a target gene encoding a protein isolated on the basis of the protein being (a) involved in an interaction of interest, (b) having a cellular localization of interest, (c) having a differential expression pattern of interest, or (d) being post-translationally modified;
(ii) identifying agents by their ability to alter the level of expression of the target gene or the activity of an expression product of the target gene;
(iii) conducting therapeutic profiling of agents identified in step (b), or further analogs thereof, for efficacy and toxicity in animals; and
(iv) formulating a pharmaceutical preparation including one or more agents identified in step (iii) as having an acceptable therapeutic profile.

23. The method of claim 22, including an additional step of establishing a distribution system for distributing the pharmaceutical preparation for sale, and may optionally include establishing a sales group for marketing the pharmaceutical preparation.

24. A method of conducting a proteomics business, comprising:
(i) by the method of claim 1 or 15, determining the identity of a target gene encoding a protein isolated on the basis of the protein being (a) involved in an interaction of interest, (b) having a cellular localization of interest, (c) having a differential expression pattern of interest, or (d) being post-translationally modified;
(ii) (optionally) conducting therapeutic profiling of the target gene for efficacy and toxicity in animals; and
(iii) licensing, to a third party, the rights for further drug development of inhibitors or activators of the target gene.
